(19) United States
(12) Patent Application Publication
(10) Pub. No.: US 2003/0162732 A1
(43) Pub. Date: Aug. 28, 2003

(54) Combination of aminosugars and cysteine or cysteine derivatives

(75) Inventor: Morten Sloth Weidner, Virum (DK)

Correspondence Address: BIRCH STEWART KOLASCH & BIRCH, PO BOX 747, FALLS CHURCH, VA, 22040-0747, US

(73) Assignee: Astion Development A/S, Kobenhavn (DK)

(21) Appl. No.: 10/185,982

(22) Filed: Jun. 28, 2002

Related U.S. Application Data
(60) Provisional application No. 60/303,298, filed on Jul. 5, 2001.

(51) Int. Cl. A61K 31/737; A61K 31/728; A61K 31/727; A61K 31/726; C08B 37/08; C08B 37/10

(52) U.S. Cl. 514/42; 514/55; 514/54; 514/56; 536/20; 536/21; 536/53; 536/54

(57) ABSTRACT

The present invention relates to chemical complexes consisting of cysteine or derivatives of cysteine and an aminosugar as well as pharmaceutical compositions and dietary supplements comprising such complexes. The invention further relates to the use of such compositions or complexes for the preparation of a medicament or a dietary supplement in the suppression of hypersensitivity and inflammatory reactions such as rheumatic or dermatological disorders or to a method of treating such diseases by administering such compositions and complexes.

### CROSS-REFERENCE TO RELATED APPLICATION

This non-provisional application claims priority under 35 U.S.C. §119(e) on U.S. Provisional Application No. 60/303,298 filed on Jul. 5, 2001, which is herein incorporated by reference.

### FIELD OF THE INVENTION

The present invention relates to the combination of cysteine or derivatives of cysteine and an aminosugar in the form of a chemical complex or a pharmaceutical composition. The invention further relates to the combined therapeutic activity of cysteine or derivatives of cysteine and an aminosugar in the suppression of hypersensitivity and inflammatory reactions such as rheumatic or dermatological disorders. The combination of N-acetylcysteine and an aminosugar may also be used as a dietary supplement.

### BACKGROUND OF THE INVENTION

Hypersensitivity is defined as a state of altered reactivity in which the body reacts with an exaggerated immune response to a substance (antigen). Hypersensitivity may be caused by exogenous or endogenous antigens. Hypersensitivity reactions underlie a large number of diseases. Among these, allergic and autoimmune conditions are of great importance. A classification of hypersensitivity diseases is given in the textbook Clinical Medicine (Kumar, P. and Clark, M.: “Clinical Medicine”, 3rd edition, p. 147-150, 1994, Bailliere Tindall, London).

Type I hypersensitivity reactions (IgE mediated allergic reactions) are caused by allergens (specific exogenous antigens), e.g. pollen, house dust, animal dandruff, moulds, etc. Allergic diseases in which type I reactions play a significant role include asthma, eczema (atopic dermatitis), urticaria, allergic rhinitis and anaphylaxis.

Type II hypersensitivity reactions are caused by cell surface or tissue bound antibodies (IgG and IgM) and play a significant role in the pathogenesis of myasthenia gravis, Good-pasture's syndrome and Addisonian pernicious anaemia.

Type III hypersensitivity reactions (immune complex) are caused by autoantigens or exogenous antigens, such as certain bacteria, fungi and parasites. Diseases in which type III hypersensitivity reactions play a significant role include lupus erythematosus, rheumatoid arthritis and glomerulonephritis.

Type IV hypersensitivity reactions (delayed) are caused by cell or tissue bound antigens. This type of hypersensitivity plays a significant role in a number of conditions, e.g. graft-versus-host disease, leprosy, contact dermatitis and reactions due to insect bites.

Type I to type IV hypersensitivity reactions are all classically allergic reactions, which may lead to histamine release. However, hypersensitivity reactions are also those, where histamine release is triggered through the directly action of “triggering substances” with the cellular membrane. Examples of “triggering substances” are, but not limited to, toxins, food constituents and certain drugs.

A number of drug classes are available for the treatment of hypersensitivity reactions. Among these, the corticosteroids are some of the most widely used drugs. Corticosteroids primarily exert their pharmacological action by non-selectively inhibiting the function and proliferation of different classes of immune cells resulting in suppression of hypersensitivity reactions. Unfortunately, the corticosteroids are associated with a number of serious side effects, e.g. immunosuppression, osteoporosis and skin atrophy.

Cancer is caused by an uncontrolled proliferation of cells that express varying degrees of fidelity to their precursors. These cancer cells form a malignant tumour that enlarges and may spread to adjacent tissues or through blood and lymph systems to other parts of the body. There are numerous forms of cancer of varying severity. For most types of cancer there is no effective treatment today.

N-acetylcysteine is a drug substance, which has been widely used as a mucolytic and as an antidote to acetaminophen poisoning.

Aminosugars are the building blocks for the in vivo generation of glycosaminoglycans, formerly known as mucopolysaccharides. Glycosaminoglycans are constituents in various tissues in numerous mammals, both vertebrates and invertebrates and important examples of glycosaminoglycans are the chondroitin sulfates and the keratan sulfates in connective tissue, the dermatan sulfates in skin tissue, and hyaluronic acid in skin tissue and synovial joint fluid.

Administration of aminosugars or glycosaminoglycans in pharmacological doses to individuals suffering from osteoarthritis has resulted in some relief of symptoms and nowadays the use of aminosugars as chondroprotective agents is widely recognised.

Glucosamines, cysteine and N-acetylcysteine are widely used for various purposes. In a number of uses they form parts of multi-agent compositions intended for use as an orally administered dietary supplement. For example, U.S. Pat. No. 5,895,652 relates to a composition comprising antioxidants, vegetable extracts, vitamins, amino acids, transition metals, fatty acids, cholinergic complexes, and enzymes as well as of N-acetylcysteine, N-acetyl-glucosamine, glucosamine and Vitamin C. Such compositions are for use as a oral administered supplement so as to augment the constituents of the cellular soup.

Furthermore, WO/00 07607 relates to a cancer-protective and cancer therapeutic composition, which comprises a large number of various constituents. The essential constituents relate to antioxidants (plant extract comprising bioflavonoids and/or vitamin C), a neovascular regulator that is an inhibitor of angiogenesis, and zinc. Such compositions may further comprise soy isolate, chondroitin sulphate and N-acetylcysteine.

The combination of aminosugars and N-acetylcysteine are also included in multi-component compositions for use in treating skin conditions. For example, U.S. Pat. No. 5,804,594 relates to orally administered compositions comprising the essential constituents: a sugar compound that is converted to a glycosaminoglycan in vivo, an antioxidant, at least one amino acid and a transition metal. Such compositions may further comprise N-acetylglucosamine, N-acetylcysteine together with amino acids, a vitamin E source, quercetin dihydrate (a bioflavonoid), a vitamin B3 source, pyridoxal 5 phosphate-Co vitamin B6, a methionine source and a vitamin A source. The skin conditions relate to wrinkles, fine lines, thinning, reduced skin elasticity, reduced skin moisture, spider veins, senile purpura, sun damaged skin, aged skin or rough skin.

Moreover, compositions for the treatment of cellulitis are disclosed. As may be understood from WO 01/13865 essential constituents of compositions for use in the treatment of cellulitis relate to a sugar compound that is converted to a glycosaminoglycan in vivo, an antioxidant (Vitamin C), at least one amino acid, a transition metal and a fat burner or a vascular dilator. Such compositions may further comprise N-acetylcysteine, N-acetylglucosamine and chondroitin sulphate.

U.S. Pat. No. 6,231,889 relates to compositions for use in the treatment of Herpes Simplex comprising a thiol-containing glutathione-increasing agent, a lysine increasing agent, a glucosamine increasing agent and magnesium.

WO 99/55326 discloses a composition comprising N-acetyl cysteine and vitamin C intended for the elevation of glutathione levels in the mammalian cell. This composition may further comprise N-acetyl-d-glucosamine.

WO 02/04003 relates to a composition comprising as an active agent, apocyanin (4-hydroxy-3-methoxy-acetophenone) or its analogues, for the treatment of arthritic conditions. This composition may further comprise N-acetylcysteine and a source of glucosamine.

WO 95/07103 relates to compositions for improved treatment of cold, cold-like and/or flu symptoms comprising as an active agent an amino acid salt of a propionic acid non-steroidal anti-inflammatory agent along with at least one decongestant, expectorant, an antihistamine and an antitussive agent. This composition may comprise N-acetylcysteine and the amino salt may be a salt including glucosamine.

U.S. Pat. No. 5,972,999 discloses a pharmaceutical composition comprising glycosaminoglycans, antioxidants, amino acids and metals for the thickening and treatment of skin conditions.

EP 93308852.8 discloses a material, which has the ability to affect the transport of other materials through the blood-brain barrier, comprising the combination of selected sugars and amino acids.

WO 93/16087 discloses Amadori reaction compounds made from selected aminosugars and amino acids. The Amadori compounds are used to produce pharmaceutical preparations, which may possess immunological activities.

Existing immunomodulating agents, such as corticosteroids or non-steroidal anti-inflammatory drugs are all known to possess good therapeutic effect on individuals suffering from diseases related to inflammatory reactions and hypersensitivity. However, as also well known these types of medication are associated with serious side effects. The present inventor sought new effective immunomodulating agents with minimal, if any, side effects.

### SUMMARY OF THE INVENTION

The present inventor has found that a combination of cysteine or cysteine derivatives and an aminosugar has immunomodulating activities and significantly suppresses inflammatory reactions and hypersensitivity in mammals. Such a combination is advantageously provided in the form of a chemical complex consisting of one or more cysteine derivative(s) of Formula I or salt(s) thereof and one or more optionally substituted aminosugar(s) or salt(s) thereof. Obviously, the combination may also be provided in the form of a pharmaceutical composition, a dietary supplement or a cosmetic. As was further recognised by the present inventor, the aminosugar according to the present invention may be an aminosugar derivative of monosaccharides, oligosaccarides as well as of polysaccharides.

Thus, the present inventor has recognised the therapeutic activity of a combination of N-acetylcysteine(s) and optionally substituted aminosugar(s), for which reason the said combination may be regarded as an active therapeutic agent.

Contrarily to existing therapeutic agents, such as corticosteroids or non-steroidal anti-inflammatory drugs, the chemical complexes and compositions according to the present invention have the advantage of not being likely to be associated with any serious side effects, as all of their components are known to living organisms and are acknowledged reported as non-toxic and well-tolerated by the organism. The present inventor puts forward the hypothesis that the very beneficial therapeutic index exhibited by the complex and compositions comprising said complex according to the invention is superior to the use of the individual constituents of the complex, and this is due to synergistic effects and a lower toxic load.

Accordingly, the present invention provides in a first aspect a chemical complex consisting of:

i) one or more cysteine derivative(s) of Formula I or salt(s) thereof; and

ii) one or more optionally substituted aminosugar(s) or salt(s) thereof.

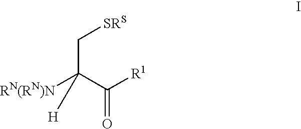

wherein R^{N} is independently selected from the group consisting of hydrogen, optionally substituted C₁-C₈-alkyl, optionally substituted C₂-C₁₀-alkenyl, optionally substituted C₂-C₁₀-alkynyl, optionally substituted C₃-C₇-cycloalkyl, and optionally substituted C₁-C₈-acyl;

R¹ is selected from the group consisting of OR3, SR3, halogen and N(RN)RN; and

R^{S} is selected from the group consisting of hydrogen, sulphate, optionally substituted C₁-C₆-alkyl, optionally substituted C₁-C₆-alkenyl, optionally substituted C₂-C₆-alkynyl, optionally substituted C₁-C₈-acyl, optionally substituted C₃-C₇-cycloalkyl, a cysteine derivative according to Formula I, and two or more cysteine derivative(s) of Formula I linked by S—S linkages.

As stated, the combination of N-acetylcysteine or derivatives or salts thereof and an aminosugar may be regarded as a therapeutic agent. Therefore, each individual compound of said combination (therapeutic agent) is essential ingredients in compositions providing immunomodulating activity. As recognised by the present inventor, a number of dietary supplements are essential for the living organism, e.g. amino acids, vitamins and transition elements. However, according to the present invention such dietary supplements are of minor importance for the providing of the immunomodulating activity according to the invention.

As follows, a further aspect of the invention relates to a composition comprising:

i) one or more cysteine derivative(s) of Formula I or salt(s) thereof; and

ii) one or more optionally substituted aminosugar or salts thereof; and

iii) one or more acceptable excipient(s) or carrier(s),

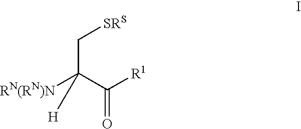

wherein R^{N} is independently selected from the group consisting of hydrogen, optionally substituted C₁-C₈-alkyl, optionally substituted C₂-C₁₀-alkenyl, optionally substituted C₂-C₁₀-alkynyl, optionally substituted C₃-C₇-cycloalkyl, and optionally substituted C₁-C₈-acyl;

R¹ is selected from the group consisting of OR3, SR3, halogen and N(RN)RN; and

R^{S} is selected from the group consisting of hydrogen, sulfate, optionally substituted C₁-C₆-alkyl, optionally substituted C₁-C₆-alkenyl, optionally substituted C₂-C₆-alkynyl, optionally substituted C₁-C₈-acyl, optionally substituted C₃-C₇-cycloalkyl, a cysteine derivative according to Formula I, and two or more cysteine derivative(s) of Formula I linked by S—S linkages, with the proviso that the composition is essentially free of Vitamin C.

As mentioned, the said complexes and compositions of the invention possess immunomodulating activities. Therefore, such chemical complexes and compositions according to the invention may be employed in a number of therapeutic areas of which the treatment of diseases related to hypersensitivity and inflammation is of importance, e.g. treatment of inflammation in skin; treatment of inflammation of joints and muscles; treatment of rheumatic diseases; IgE mediated allergic reactions and conditions; autoimmune disorders; alleviation of pain; or cancer.

An important aspect of the invention relates to the use of a combination of one or more cysteine derivative(s) of Formula I or salt(s) thereof and one or more optionally substituted aminosugar(s) or salt(s) thereof for the preparation of a product for the suppression of hypersensitivity and/or suppression of inflammatory reactions in a mammal, as well as to a method for suppression of hypersensitivity and suppression of inflammatory reactions in a mammal, comprising the administration to said mammal of an effective amount of a combination of one or more cysteine derivative(s) of Formula I or salt(s) thereof and one or more optionally substituted aminosugar(s) or salt(s) thereof, or a chemical complex comprising said combination.

Still further aspects of the invention relate independently to a method for the treatment of hypersensitivity skin disease in a mammal; a method for the treatment; a method for the treatment or prevention of IgE mediated allergic reaction and/or condition in a mammal; a method for the treatment of an autoimmune disease and/or a chronic inflammatory disease in a mammal; a method for the treatment of rheumatic diseases; a method for the alleviation of pain in a mammal, and a method for the treatment or prevention of cancer in a mammal each method comprising the administration to said mammal of an effective amount of a combination of one or more cysteine derivative(s) of Formula I or salt(s) thereof and one or more optionally substituted aminosugar(s) or salt(s) thereof, or a chemical complex comprising said combination.

### DETAILED DESCRIPTION OF THE INVENTION

The present inventor provides data herein indicating that a combination of one or more cysteine derivative(s) of Formula I or salt(s) thereof and one or more optionally substituted aminosugar(s) or salt(s) thereof significantly reduces the inflammation in the carrageenin-induced paw oedema test in the rat. This reduction in the paw oedema was comparable to that of therapeutically relevant doses of Ibuprofen, a non-steroidal anti-inflammatory agent used in the treatment of muscle pain and rheumatic disorders (see example 282).

The carrageenin-induced paw oedema test in the rat is widely acknowledged, for example the carrageenin rat paw oedema model has been established by a workgroup under the US FDA as the best pre-clinical method to predict the effective clinical dose of non-steroidal antiinflammatory drugs in humans (Inflammation Research 45: 531-540, 1996).

Furthermore, the present inventor provides evidence that the present complexes and compositions according to the invention are effective in relieving chronic muscle pain and some degree of immobility of the arm in a person suffering from tendonitis of the arm. Moreover, efficacy against osteoarthritis of the hips and knees is shown (see example 281). In accordance with the anti-inflammatory activity seen in the carrageenin-induced paw oedema test in the rat, the combination of one or more cysteine derivative(s) of Formula I or salt(s) thereof and one or more optionally substituted aminosugar(s) or salt(s) thereof is effective in suppressing hypersensitivity and inflammatory reactions.

According to the invention, the combination of one or more cysteine derivative(s) of Formula I or salt(s) thereof and one or more optionally substituted aminosugar(s) or salt(s) thereof may be provided in the form of a chemical complex, in the form of a composition comprising said complex and optionally pharmaceutically acceptable excipient(s) or in the form of a pharmaceutical composition comprising the combination of the of one or more cysteine derivative(s) of Formula I or salt(s) thereof and one or more optionally substituted aminosugar(s) or salt(s) thereof. Moreover, the one or more cysteine derivative(s) of Formula I or salt(s) thereof and the one or more optionally substituted aminosugar(s) or salt(s) thereof may each be provided in separate compositions.

Without being limited to a particular theory, advantageously, said combination is provided in the form of a chemical complex for purposes of achieving a homogeneous mixture of the two agents, which may positively affect the resulting therapeutic effect.

Such chemical complexes are novel and provide a surprisingly effective anti-hypersensitivity and anti-inflammatory effect with a surprisingly good safety profile. Thus the chemical complexes or compositions of the invention are virtually non-toxic and yet very therapeutically effective.

The present inventor proposes the hypothesis that the very advantageous therapeutic index of said combinations of the one or more cysteine derivative(s) of Formula I or salt(s) thereof and the one or more optionally substituted aminosugar(s) or salt(s) thereof in comparison to their individual anti-inflammatory effect is due to the synergistic effects between the components of the compositions. Therefore, lower doses may be needed for providing the therapeutic effect, resulting in a lower toxic load on the body in comparison to the individual compound, while still achieving a surprisingly good therapeutic effect.

Accordingly, the present invention provides in a first aspect a chemical complex consisting of:

i) one or more cysteine derivative(s) of Formula I or salt(s) thereof; and

ii) one or more optionally substituted aminosugar(s) or salt(s) thereof.

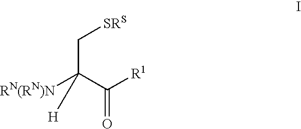

wherein R^{N} is independently selected from the group consisting of hydrogen, optionally substituted C₁-C₈-alkyl, optionally substituted C₂-C₁₀-alkenyl, optionally substituted C₂-C₁₀-alkynyl, optionally substituted C₃-C₇-cycloalkyl, and optionally substituted C₁-C₈-acyl;

R¹ is selected from the group consisting of OR3, SR3, halogen and N(RN)RN; and

R^{S} is selected from the group consisting of hydrogen, sulfate, optionally substituted C₁-C₆-alkyl, optionally substituted C₁-C₆-alkenyl, optionally substituted C₂-C₆-alkynyl, optionally substituted C₁-C₈-acyl, optionally substituted C₃-C₇-cycloalkyl, a cysteine derivative according to Formula I, and two or more cysteine derivative(s) of Formula I linked by S—S linkages.

A further aspect relates to a composition comprising:

i) one or more cysteine derivative(s) of Formula I or salt(s) thereof; and

ii) one or more optionally substituted aminosugar or salts thereof; and

iii) one or more acceptable excipient(s) or carrier(s),

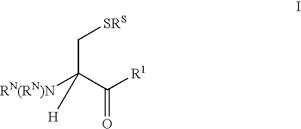

wherein R^{N} is independently selected from the group consisting of hydrogen, optionally substituted C₁-C₈-alkyl, optionally substituted C₂-C₁₀-alkenyl, optionally substituted C₂-C₁₀-alkynyl, optionally substituted C₃-C₇-cycloalkyl, and optionally substituted C₁-C₈-acyl;

R¹ is selected from the group consisting of OR3, SR3, halogen and N(RN)RN; and

R^{S} is selected from the group consisting of hydrogen, sulfate, optionally substituted C₁-C₆-alkyl, optionally substituted C₁-C₆-alkenyl, optionally substituted C₂-C₆-alkynyl, optionally substituted C₁-C₈-acyl, optionally substituted C₃-C₇-cycloalkyl, a cysteine derivative according to Formula I, and two or more cysteine derivative(s) of Formula I linked by S—S linkages, with the proviso that the composition is essentially free of Vitamin C,

In one embodiment thereof, the composition comprises the one or more cysteine derivative(s) of Formula I or salts thereof and the one or more optionally substituted aminosugar or salts thereof in the form of a chemical complex.

The term “chemical complex” is intended to include the definition defined by IUPAC that read as follows:

“A molecular entity formed by loose association involving two or more component molecular entities (ionic or uncharged), or the corresponding chemical species. The bonding between the components is normally weaker than in a covalent bond.” (IUPAC Compendium of Chemical Terminology 2nd Edition (1997))

Thus, the term “chemical complex” is intended to mean any combination of the component molecules. It is not intended necessarily to implie an ionic or otherwise association between the components. Also as used herein, the chemical complex of the present invention relates to a complex obtainable from the combining of one or more cysteine derivative(s) of Formula I or salts thereof and one or more optionally substituted aminosugar or salts thereof.

The complexes of the invention may be prepared according to a number of different methods, which are obvious to a person skilled in the art. The following procedures are non-limiting examples of such methods:

The components of the complex, dosed in appropriate amounts to give the correct molar ratio between the moieties, are dissolved, dispersed, or suspended in an appropriate solvent, for example water, an organic solvent or mixtures thereof. Non-limiting examples of suitable organic solvents are ethanol, methanol, iso-propyl alcohol, acetone, hexane, ethylacetate or mixtures thereof.

The solvent is then removed by a technique suitable for the complex, for example but not limited to evaporation, in vacou evaporation, spray drying, freeze-drying, fluid bed drying or spin flash drying. Alternatively the complex may be obtained by precipitation and subsequent centrifugation or filtering.

The term “optionally substituted” is intended to mean the substitution of one or more hydrogen atoms, which is substituted with another atom, chemical group or entity, termed substituents. Illustrative examples of substituents include carboxyl, formyl, amino, hydroxyl, halogen, nitro, sulphono, sulphanyl, C₁₋₆-alkyl, aryl, aryloxy, aryloxycarbonyl, arylcarbonyl, heteroaryl, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyloxy, C₁₋₆-alkylsulphonyloxy, dihalogen-C₁₋₆-alkyl, trihalogen-C₁₋₆-alkyl, C₁₋₆-alkoxyl, oxo, C₁₋₆-carboxyl, C₁₋₆-alkoxycarbonyl, C₁₋₆-alkylcarbonyl,

where aryl and heteroaryl representing substituents may be substituted 1-5 times with C₁₋₆-alkyl, C₁₋₆-alkoxy, nitro, cyano, hydroxy, amino or halogen. In general, the above substituents may be susceptible to further optional substitution.

The term “halogen” includes fluorine, chlorine, bromine and iodine.

In the present invention, the term “aminosugar” is intended to mean one or more amino derivatives of a monosaccharide (aldoses and ketoses) and its corresponding sugar alcohols (alditols) such as trioses, tetroses, pentoses, hexoses, heptoses and octoses. The aldose, ketose, or alditol has one or more hydroxy groups replaced by any amino group at any position, including the anomeric position. An aminosugar is thus a deoxyamino derivative of an aldose, ketose, or alditol. The term is also intended to mean polyamino sugars, wherein more than one hydroxy group has been replaced by an amino group (e.g. dideoxydiamino-, trideoxytriamino-derivatives).

Moreover, the term “aminosugar” is also intended to mean amino derivatives of di-, oligo- and poly-saccharides comprising at least one of said monosaccharides. Consequently, in the case of di-, oligo- and poly-saccharides, the amino group may be the position of glycosidation. Suitably, in di-, oligo- and poly-saccharides, the amino group may not be the position of glycosidation.

In a suitable embodiments thereof, the general structure of the aminosugar is a saccharide chain comprising a linkage region, a chain cap and a repeat region. In one embodiment, the repeat region comprises at least one disaccharide unit in which one or both of the sugar monomers of said disaccharide unit is either galactosamine or glucosamine, such as N-acetylgalactosamine or N-acetylglucosamine; the linkage region that is linked the repeat region is present at least once and may be a di-, oligo or poly-saccharide or a di-, oligo or poly-saccharide chain with a terminal amino acid; and is suitable for linking to a protein; and the cap region is a di-, oligo or poly-saccharide present at least once and is linked to the repeat region.

The other sugar monomer of said disaccharide-repeating region might be selected from the array of hexoses known to the person skilled in the art. Illustrative examples of preferred embodiments of monomers include D-glucuronic acid, L-iduronic acid, D-galacturonic acid, D-galactose, and fucose, each of which may be optionally sulfonated or O-substituted with a protective group known to the person skilled in the art.

The number of repeat units in an aminosugar of the invention may range from 1 to 500000, such as from 2 to 50000, preferably from 2 to 10000, most preferably from 2 to 1000. In suitable embodiments, the aminosugar comprises of 30 to 50 disaccharide units.

According to the invention preferred repeat regions may be selected from Formula II, III, IV, V, VI, VII or VIII, which are non-limiting examples. Accordingly, the aminosugar may comprise of a repeat unit comprising of any combination of disaccharides according to Formula II to VIII.

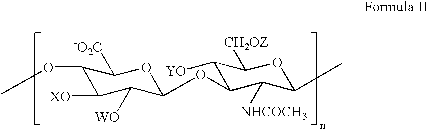

β-D-glucuronic acid N-acetyl-β-D-glucosamine, wherein W, X, Y, and Z independently may be SO₃⁻ or H.

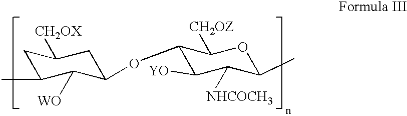

β-D-galactose N-acetyl-β-D-glucosamine, wherein W,X,Y and Z independently may be SO₃⁻ or H.

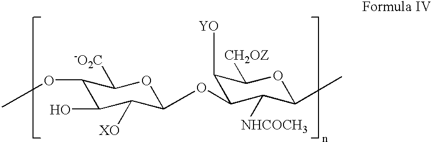

β-D-glucuronic acid N-acetyl-β-D-galactosamine, wherein X, Y and Z independently may be SO₃⁻ or H.

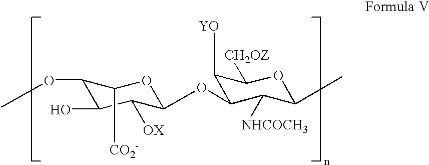

α-L-iduronic acid N-acetyl-β-D-galactosamine, wherein X, Y and Z may independently be SO₃⁻ or H.

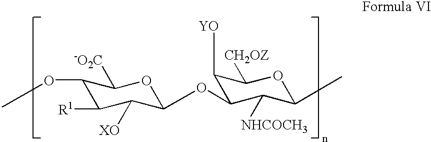

β-D-glucuronic acid N-acetyl-β-D-galactosamine, wherein R¹ may be fucose or OSO₃ or OH and X, Y and Z independently may be SO₃⁻ or H.

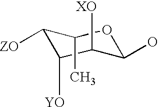

Fucose, wherein X, Y and Z independently may be SO₃⁻ or H.

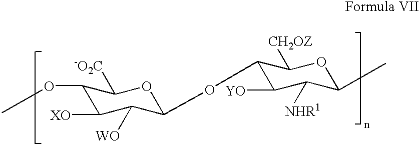

β-D-glucuronic acid D-glucosamine, wherein W, X, Y and Z independently may be SO₃⁻ or H and R¹ may be COCH₃ or SO₃⁻.

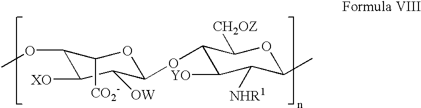

α-L-iduronic acid D-glucosamine, wherein W, X, Y and Z independently may be SO₃⁻ or H and R¹ may be COCH₃ or SO₃⁻.

As stated, the number of repeat units in an aminosugar according to the invention may range from 1 to 500000. Accordingly the value of n, or the sum of n for all of the disaccharide units according to any of Formula I-VIII (n₂+n₃+n₄+n₅+n₆+n₇+n₈), may be in the range of 1 to 500000, such as from 2 to 50000, preferably from 2 to 10000, most preferably from 2 to 1000. In suitable embodiments, the aminosugar comprises of 30 to 50 disaccharide units, i.e. n may be in the range.

D-glucuronic acid is the principle uronic acid present in hyaluronic acid and chondroitin sulfates A and C, while dermatan sulfate (chondroitin sulfate B) contains L-iduronic acid. In keratan sulfate the principle uronic acid is D-galacturonic acid. In all of the aminosugars the disaccharide unit may be non-sulfated, mono-sulfated, di-sulfated or tri-sulfated, and different uronic acids may be present in a given polymer chain.

The linkage region is the moiety of the aminosugar, which may be O-linked to a protein in the use of the chemical complex in the treatment defined herein. The linkage unit may be a di-, oligo or poly-saccharide and is linked to at least one repeat region. Linkage to a protein may be from any of the oxygen atoms of the terminal saccharide of the linkage unit. The linkage region may be linked to any part of the repreating region. Typically, the linkage region will be at the terminus of the repeating region.

Alternatively, the linkage region comprises a di-, oligo or poly-saccharide chain with a terminal amino acid. It such an embodiment, linkage to a protein is via said amino acid. In a preferred embodiment, the amino acid is serine.

An aminosugar may be linked more than once to a protein and there may be more than one linkage region. In a suitable embodiment, there is one linkage region hence the aminosugar is linked once to a protein. Conversely, more than one glucoaminoglycan may be linked to a single protein.

In a preferred embodiment, the linkage region is of the general form:

-4(GlcAβ(1-3)Galβ(1-3)Galβ(1-4)Xylβ(1-0)-Ser, where in C-4 and C-6 in Galβ independently may be SO₃ or OH.

(Abbreviations: GICA, glucuronic acid; Gal, galactose; Xyl, xylose; Ser, serine.)

In the embodiment wherein the aminosugar is chondroitin sulfate, said glucosaminoglycan is preferably O-linked to a serine of a protein core.

The chain cap is the chain terminus in the aminosugar. Thus, the chain cap comprises of a mono-, di- or oligo-saccharide at one or both of the two termini of the repeating regions of the aminosugar. In the typical embodiment wherein the linkage region is at the terminus of the repeat region, there will only be one chain cap.

The chain cap will typically be in the form of N-acetyl-β-D-galactosamine or N-acetyl-β-D-glucosamine, which may be non-sulfated, mono-sulfated or di-sulfated.

In a preferred embodiment, the chain cap will have a lower degree of sulfonation than the repeating region. The saccharide or saccharides of the chain may be O-protected in the manner known to the skilled artisan.

In the event wherein the aminosugar is chondroitin sulfate, the chain cap may be in the form of formula 8

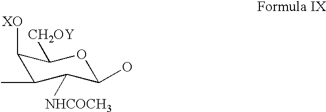

N-acetyl-p-D-galactosamine, wherein X and Y independently may be SO₃⁻ or OH.

Glucosaminoglycans, formerly known as mucopolysaccharides, are components of various tissues in numerous animals, both vertebrates and invertebrates. All are polymers of repeating disaccharide units, in which one of the sugars is either N-acetylgalactosamine or N-acetylglucosamine. Important examples are the hyaluronic acid, chondroitin sulfates and keratan sulfates of connective tissue, the dermatan sulfates (chondroitin sulfates B) of skin, and heparin and heparan sulfates of mast cells.

A major function of some glycosaminoglycans is the formation of a matrix to hold together the protein components of the skin and connective tissue.

The filamentous structure is built on a single hyaluronic acid molecule, to which extended core proteins are attached noncovalently. These, in turn, have chondroitin sulfate and keratan sulfate chains covalently bound to them though serine side chains. In cartilage, this kind of structure binds collagen and helps hold the collagen fibres in a tight, strong network. The binding apparently involves electrostatic interactions between the sulfate and/or carboxylate groups and basic side chains in collagen.

The aminosugars of the present invention may be obtained from any such biological glucosaminoglycan-containing material by any available method obvious to a person skilled in the art, e.g. chemical and/or mechanical processing. Thus according to the invention glycosaminglycan polymers can be depolymerised, for example by a number of specific enzymes or by acid hydrolysis to give low molecular weight species, which carry a range of negative charges depending on the number of sulphated groups attached. A number of relevant enzymes are available such as chondroitinases, keratanases, hyaluronidases, heparinases, heparitinases etc. There are a number of subtypes of such enzymes with selective activity making it possible to manufacture numerous fragments of glucosaminoglycans, which can be used according to the invention. The molecular weight of such fragments is preferably in the range from 5000 to 1000000 Da, e.g. from 6000 to 500000 Da, such as from 7000 to 300000 Da, even more preferably from 8000 to 200000 Da, such as from 9000,to 100000 Da, e.g. from 10000 to 50000 Da. In another preferred embodiment of the invention the molecular weight of the glucosaminoglucan fragment is below 5000 Da and even more preferably below 3000 Da.

An amino group of an aminosugar may be alkylated, arylated or acylated or, alternatively, present as its free amine form (NH2). Similarly, the hydoxyl groups may be optionally protected or derivatised such as alkylated, arylated or acylated or, alternatively, present in its free hydroxyl form.

The amine of the amino sugar may exist as its ammonium salt using organic or mineral acids, as is known to the person skilled in the art. However, according to the invention, the ammonium salt is preferably of a mineral acid and an aminosugar. Furthermore, other functional groups on the aminosugar may be in the form of a salt. Similarly, prodrug derivatives of the aminosugar are anticipated by the present inventor. The prodrug form may be the result of the derivatisation of the amino group or another functional group present on the aminosugar, as is known to the person skilled in the art.

Furthermore, an aminosugar may have one or more hydroxy groups replaced by any amino group at any position and a further one or more hydroxy groups replaced by a hydrogen (a deoxy sugar), a thiol (a thiosugar), a halogen (a deoxyhalo sugar), an anhydrosugar (a sugar preparable via an intramolecular displacement with a hydroxyl to form an oxirane or oxetane), a carbonyl group.

In a particularly suitable embodiment of the invention, the aminosugar is sulphated or phosphorylated at the anomeric, 2-, 3-, 4-, or 6-position, typically at the 2-, 3-, or 4-position. In another suitable embodiment of the invention the aminosugar is N-acetylated.

Furthermore, a combination of suitable embodiments include the aminosugar sulphated or phosphorylated as well as in its salt form having Na⁺; K⁺; Mg++; Ca++; or NH4+ as counter ions.

Particularly suitable aminosugars according to the invention are glucosamine, galactosamine or mannosamine, their derivatives and salts thereof, typically glucosamine sulfate, glucosamine hydrochloride, N-acetylglucosamine, galactosamine sulfate, galactosamine hydrochloride, N-acetylgalactosamine, mannosamine sulfate, mannosamine hydrochloride or N-acetylmannosamine. Also other aminosugars known to the person skilled in the art are suitable for use.

In a suitable embodiments, di-, oligo-, and poly-saccharides comprising at least one aminosugar is comprised in the complex. In the embodiment wherein the aminosugar is an oligo- or polysaccharide, said oligo- or polysaccharide preferably consists of more than one aminosugar monosaccharide. Suitable sources of the aminosugar may be chitin, chitosan, carboxymethyl-chitosan, chondroitin sulfate, keratan sulfate dermatan sulfates and hyaluronic acid.

As stated, the complex and compositions of the invention also comprises one ore more cysteine derivative(s) of Formula I or salts thereof.

As used herein the term “cysteine derivative” is intended to mean cysteine and a cysteine, wherein the hydrogen of the cysteine may be replaced by a substituent. Thus, in a suitable embodiment, the cysteine derivative may therefore be of the general Formula I,

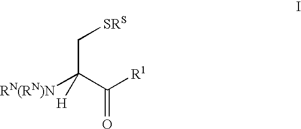

wherein R^{N} independently selected from the group consisting of hydrogen, optionally substituted C₁-C₈-alkyl, optionally substituted C₂-C₁₀-alkenyl, optionally substituted C₂-C₁₀-alkynyl, optionally substituted C₁-C₈-acyl, optionally substituted aryl, optionally substituted heteroaryl, optionally substituted heterocycle, optionally substituted C₃-C₇-cycloalkyl, CH₂—N(R3)(R3), CH₂—OR3, CH₂—SR3, CH₂—O—C(═O)R3, CH₂—O—C(═O)—OR3, CH₂—O—C(═S)R3, CH₂—S—C(═O)R3, C(═O)(R3), C(═S)R3, —C(═S)—OR3, —C(═O)—SR3, C(═O)—N(R3)(R3), and C(C═S)—N(R3)(R3), wherein R3 is selected from the group consisting of hydrogen, optionally substituted C₁-C₆-alkyl, optionally substituted C₁-C₆-alkenyl, optionally substituted C₂-C₆-alkynyl, optionally substituted aryl, optionally substituted heteroaryl, optionally substituted heterocycle, and optionally substituted C₃-C₇-cycloalkyl;

R1 is selected from the group consisting of OH, OR3, SR3, halogen and N(RN)RN; and

RS is selected from the group consisting of hydrogen, optionally substituted C₁-C₆-alkyl, optionally substituted C₁-C₆-alkenyl, optionally substituted C₂-C₆-alkynyl, optionally substituted C₁-C₈-acyl, optionally substituted aryl, optionally substituted heteroaryl, optionally substituted heterocycle, optionally substituted C₃-C₇-cycloalkyl, a cysteine derivative according to Formula I, and two or more cysteine derivative(s) of Formula I linked by S—S linkages.

Without being limited to a particular theory, it is the current understanding that the thiol group of the cysteine moiety is important for the activity of the complex. Thus, typically, the thiol group is not derivatized, i.e. RS is hydrogen. As it would be known to the person skilled in the art, even within the current theory, RS may be such that, upon administration, in vivo hydrolysis of RS would provide the free thiol SH.

Moreover, the chemical complexes and compositions of the present invention may comprise cysteine derivative precursors, which upon administration and in vivo chemical modification or enzymatic modification, provide a derivative of cysteine according to formula 1. Thus, in one embodiment, the chemical complexes and compositions comprise N-acetylated cysteine or a N-acetylated cysteine derivative that are deacetylated in vivo to form cysteine and a cysteine derivative, respectively.

Suitable embodiments of cysteine derivatives of formula I may be the N-acetyl derivative, as discussed supra, but also be other cysteine derivatives such as the free amine (NH2, wherein both RN groups are hydrogen), the N-benzyl, N-benzoyl, other N-acyl derivatives and N-alkyl derivatives. Embodiments wherein RN results in a prodrug such that the free amine is generated in vivo is a particularly interesting aspect of the invention. Cysteic acid and cystine are alternative putative sources of cysteine in vivo. Similarly, the free amine or quaternary ammonium salts of the amine of compounds of formula II are interesting embodiments of compounds of formula II, such as cysteine hydrochloride.

Suitable embodiments of compounds of formula I are such that SRS is HOOC—CH2-S, as in carboxymethyl cysteine (carbocysteine), metcysteine and isobutyryl cysteine. In a further embodiment of the invention, the complex may consist of homocysteine, or a derivative thereof, and an aminosugar. Correspondingly, the present inventor anticipates that the complex may consist of methionine, or a derivative thereof, and an aminosugar.

The term “cysteine derivative” is furthermore intended to mean cysteine dimers, oligomers, and polymers, such as peptides and polypeptides of cysteine wherein the N-terminal end is preferably acetylated.

In a preferred embodiment of the invention, the one or more cysteine derivative(s) of Formula I is N-acetylcysteine. N-acetyicysteine may be obtained from natural sources or synthetically. However, N-acetylcysteine may also be obtained from precursors, which upon chemical or enzymatic reactions release free N-acetylcysteine. Such chemical or enzymatic release from precursors of N-acetylcysteines may take place either in vivo after administering the precursor or outside the body. A particularly suitable example of a potential precursor is cysteine itself, which may be acetylated by bacteria in the gut lumen or enzymatically during the penetration of the gut wall into the systemic circulation. Cysteine may be acetylated to N-acetylcysteine in a pharmaceutical formulation containing acetylating bacteria, e.g. E. coli bacteria and lactic bacteria.

In typical embodiments of the invention, the cysteine derivative(s) of Formula I or salt(s) is cysteine, Na-acetylcysteine, cystine, homocysteine, cysteine methylester, S-ethyl-cysteine, N,S-isobuturyl-cysteine, S-carboxymethyl-cysteine, S-ethyl-homocysteine, S-methyl-cysteine, Cysteine S-sulfate, N,S-diacetyl-cysteine methylester, N-acetyl-S-methylcysteine or salts thereof.

As stated the combination of the two kinds of compounds provides a surprisingly effective therapeutic agent for suppression of hypersensitivity and inflammatory reactions. The proper therapeutic efficacy may, in part, be adjusted by providing the two agents in suitable molar ratios or mass ratios.

Hence, the combination of the one or more cysteine derivative(s) of Formula I or salt(s) thereof and the one or more optionally substituted aminosugar(s) or salt(s) thereof in a chemical complex or in a composition according to the invention are present in a molar ratio of between about 1:10000 to 10000:1. Preferably, the molar ratio is of between about 1:1000 to 1000:1, 1:500 to 500:1, or about 1:100 to 100:1, preferably of about 1:50 to 50:1, such as about 1:25 to 25:1, about 1:20 to 20:1, about 1:18 to 18:1, about 1:16 to 16:1, about 1:14 to 14:1, or about 1:12 to 1:12, more preferably of about 1:10 to 10:1, such as about 1:9 to 9:1, about 1:8 to 8:1, about 1:7 to 7:1, about 1:6 to 6:1, such as from 1:5 to 5:1, such as from 1:4 to 4:1, from 1:3 to 3:1, such as from 1:2 to 2:1, such as 1:1.

Alternatively defined, the ratio between one or more cysteine derivative(s) of Formula I or salt(s) thereof and the one or more optionally substituted aminosugar(s) or salt(s) thereof may be expressed as a mass ratio. The mass ratio is of between about 1:10000 to 10000:1. Preferably, the mass ratio is of between about 1:1000 to 1000:1, 1:100 to 100:1, 1:50 to 50:1, or about 1:40 to 40:1, preferably of about 1:30 to 30:1, such as about 1:25 to 25:1, about 1:20 to 20:1, about 1:18 to 18:1, about 1:16 to 16:1, about 1:14 to 14:1, or about 1:12 to 1:12, more preferably of about 1:10 to 10:1, such as about 1:9 to 9:1, about 1:8 to 8:1, about 1:7 to 7:1, about 1:6 to 6:1, such as from 1:5 to 5:1, such as from 1:4 to 4:1, from 1:3 to 3:1, such as from 1:2 to 2:1, such as 1:1.

For the administration to a mammal, such as a human, the chemical complex may be administered directly, eventually provided in a capsule or the like. More convenient, the complex may be formulated into a composition comprising the chemical complex and optionally, one or more acceptable excipients. Alternatively, the combination of the two agents may also be formulated into a composition without being provided as a chemical complex. Thus, in some embodiments of the invention, the chemical complexes or compositions further comprise one of more excipent(s) or carrier(s), preferably pharmaceutically acceptable excipent(s) or carrier(s).

The term “composition” is intended to mean cosmetic compositions, pharmaceutical compositions, nutritional compositions such as food supplements as well as compositions in the field of cosmeceuticals and neutraceuticals.

As stated supra, the combination of the one or more cysteine derivative(s) of Formula I or salt(s) thereof and the one or more optionally substituted aminosugar(s) or salt(s) thereof possesses significant anti-hypersensitivity and anti-inflammatory activity. Accordingly, said combination is the active agent in compositions for use in the treatment of diseases or disorders associated with inflammation and/or hypersensitivity. For that reason, the compositions of the present invention does not necessarily comprise other compounds than those excipients needed for the formulation of a pharmaceutical or dietary supplement. That is to say that a number of compounds are not considered to add potential benefits to the composition of the invention or to the use according to the present invention of said compositions for the suppression of hypersensitivity and inflammation.

Hence in one embodiment of the invention, the composition consists of one or more cysteine derivative(s) of Formula I or salt(s) thereof and one or more optionally substituted aminosugar(s) or salt(s) together with one or more acceptable excipient(s) or carrier(s).

Moreover, according to the invention the compositions may be essentially free of dietary constituents that forms part of the daily food intake, e.g. various vitamins, antioxidants, transition metals and the essential amino acids. Accordingly, in one embodiment the compositions of the invention are essentially free of vitamin C, and if possible they do not contain vitamin C. In a further embodiment thereof, the compositions of the invention are essentially free of or do not contain one or more of the essential amino acids such as lysine, for example in the form of lysine monohydrochloride. Furthermore, it is not intended to include compounds extracted from natural sources in the composition. Thus, in a further embodiment of the invention, the compositions do not contain plant extracts, e.g. plant extracts comprising bioflavonoids. For example, quercetin is a bioflavonoid normally present in plant extracts. In still further embodiments, the compositions do not contain plant extracts or do not contain bioflavonoides. Apocynine is another compound present in plant extracts. Accordingly, in further embodiments the compositions do not contain apocynine or its analoques.

As stated, the compositions of the invention comprise acceptable excipients. In one embodiment of the invention, the acceptable excipients does not include a magnesium salt in general or in particular magnesium ascorbate, magnesium L-acetylcysteinate, magnesium N-thioctyltaurate, magnesium taurate, magnesium citrate and/or magnesium oxide. That is to say that the composition is essentially free of or does not contain a magnesium salt selected from the group consisting of magnesium ascorbate, magnesium L-acetylcysteinate, magnesium N-thioctyltaurate, magnesium taurate, magnesium citrate and magnesium oxide.

The chemical complexes or compositions of the present invention may be combined with any other therapeutically active agent in order to strengthen, improve, potentiate, or prolong the therapeutic actions of said complexes and said compositions. Thus according to the invention, the composition may further comprise one or more suitable therapeutically active agent, e.g. an agent for treating cancer. For the fact that the present combination has anti-inflammatory effect, it is not the intention of adding any other anti-inflammatory drug such as for example non-steroidal anti-inflammatory agent or an antihistamine. Thus, in one further embodiment, the composition does not comprise another anti-inflammatory agent selected from the group of non-steroidal anti-inflammatory agents and antihistamines.

As stated, the complexes and compositions according to the invention may comprise one or more aminosugar(s). Thus, a mixture of various aminosugars is anticipated. However, in some embodiments, the mixtures contains low molecular aminosugars, such as aminosugar derivatives of mono-saccharides. In other embodiments, the aminosugar(s) consists only of aminosugar derivatives of polysaccharides, such as chondroitin, heparin and the like. Thus, in some interesting embodiments of the invention, the mixtures does not comprise the mixture of a glucosamine and a chondroitin.

The compositions according to the present invention may be formulated as a pharmaceutical composition for oral, topical, transdermal, or parenteral administration, preferably oral or topical administration.

In a suitable embodiment of the invention, the compositions are used for oral administration. In another suitable embodiment of the invention the compositions are used for topical administration. The pharmaceutical compositions for oral, topical, transdermal, or parenteral administration may be in form of, e.g., solid, semi-solid or fluid compositions and formulated according to conventional pharmaceutical practice, see, e.g., “Remington: The science and practice of pharmacy” 20^{th} ed. Mack Publishing, Easton Pa., 2000 ISBN 0-912734-04-3 and “Encyclopedia of Pharmaceutical Technology”, edited by Swarbrick, J. & J. C. Boylan, Marcel Dekker, Inc., New York, 1988 ISBN 0-8247-2800-9.

The choice of pharmaceutically acceptable excipients in a composition for use according to the invention and the optimum concentration thereof is determined on the basis of the selection of the cysteine derivative(s) of Formula I, selection of the aminosugar, the kind of dosage form chosen and the mode of administration. However, a person skilled in the art of pharmaceutical formulation may find guidance in e.g., “Remington: The science and practice of pharmacy” 20^{th} ed. Mack Publishing, Easton Pa., 2000 ISBN 0-912734-04-3. A pharmaceutically acceptable excipient is a substance, which is substantially harmless to the individual to which the composition will be administered. Such an excipient suitably fulfils the requirements given by the national drug agencies. Official pharmacopeias such as the British Pharmacopeia, the United States of America Pharmacopeia and the European Pharmacopeia set standards for well-known pharmaceutically acceptable excipients.

For topical, trans-mucosal and trans-dermal compositions, such as administration to the mucosa or the skin, the compositions for use according to the invention may contain conventional non-toxic pharmaceutically acceptable carriers and excipients including microspheres and liposomes.

The topical, trans-mucosal and trans-dermal compositions for use according to the invention include an array of solid, semi-solid and fluid compositions. Compositions of particular relevance are e.g. pastes, ointments, hydrophilic ointments, creams, gels, hydrogels, solutions, emulsions, suspensions, lotions, liniments, resoriblets, suppositories, enema, pessaries, moulded pessaries, vaginal capsules, vaginal tablets, shampoos, jellies, soaps, sticks, sprays, powders, films, foams, pads, sponges (e.g. collagen sponges), pads, dressings (such as, e.g., absorbent wound dressings), drenches, bandages, plasters and transdermal delivery systems.

The pharmaceutically acceptable excipients for topical, trans-mucosal and trans-dermal compositions may include solvents, buffering agents, preservatives, humectants, chelating agents, antioxidants, stabilizers, emulsifying agents, suspending agents, gel-forming agents, ointment bases, suppository bases, penetration enhancers, perfumes, skin protective agents, diluents, disintegrating agents, binding agents, lubricants and wetting agents.

The oral compositions for use according to the invention include an array of solid, semi-solid and fluid compositions. Compositions of particular relevance are e.g. solutions, suspensions, emulsions, uncoated tablets, immediate-release tablets, modified-release tablets, gastro-resistant tablets, orodispersible tablets, efferverscent tablets, chewable tablets, soft capsules, hard capsules, modified-release capsules, gastro-resistant capsules, uncoated granules, effervescent granules, granules for the preparation of liquids for oral use, coated granules, gastro-resistant granules, modified-release granules, powders for oral administration and powders for the preparation of liquids for oral use.

The pharmaceutically acceptable excipients may include solvents, buffering agents, preservatives, humectants, chelating agents, antioxidants, stabilizers, emulsifying agents, suspending agents, gel-forming agents, diluents, disintegrating agents, binding agents, lubricants, coating agents and wetting agents.

Typical solvents may be selected from the group comprising water, alcohols, vegetable or marine oils (e.g. edible oils like almond oil, castor oil, cacao butter, coconut oil, corn oil, cottonseed oil, linseed oil, olive oil, palm oil, peanut oil, poppyseed oil, rapeseed oil, sesame oil, soybean oil, sunflower oil, and teaseed oil), mineral oils, fatty oils, liquid paraffin, polyethylene glycols, propylene glycols, glycerol, liquid polyalkylsiloxanes, and mixtures thereof.

Typical buffering agents may be selected from the group comprising citric acid, acetic acid, tartaric acid, lactic acid, hydrogenphosphoric acid, diethylamine.

Typical preservatives may be selected from the group comprising parabens, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

Typical humectants may be selected from the group comprising glycerin, propylene glycol, sorbitol, lactic acid, urea, and mixtures thereof. Typical chelating agents may be selected from the group comprising sodium EDTA and citric acid. Typical antioxidants may be selected from the group comprising butylated hydroxy anisole (BHA), ascorbic acid and derivatives thereof, tocopherol and derivatives thereof and mixtures thereof. Suitable emulsifying agents may be selected from the group comprising naturally occurring gums, e.g. gum acacia or gum tragacanth; naturally occurring phosphatides, e.g. soybean lecithin; sorbitan mono-oleate derivatives; wool fats; wool alcohols; sorbitan esters; monoglycerides; fatty alcohols; fatty acid esters (e.g. triglycerides of fatty acids); and mixtures thereof.

Suitable suspending agents may be selected from the group comprising celluloses and cellulose derivatives such as, e.g., carboxymethyl cellulose, hydroxyethylcellulose, hydroxypropylcellulose, hydroxypropylmethylcellulose, carrageenan, acacia gum, arabic gum, tragacanth, and mixtures thereof.

Suitable gel bases and viscosity-increasing components may be selected from the group comprising liquid paraffin, polyethylene, fatty oils, colloidal silica or aluminium, zinc soaps, glycerol, propylene glycol, tragacanth, carboxyvinyl polymers, magnesium-aluminium silicates, Carbopol®, hydrophilic polymers such as, e.g. starch or cellulose derivatives such as, e.g., carboxymethylcellulose, hydroxyethylcellulose and other cellulose derivatives, water-swellable hydrocolloids, carragenans and alginates including propylene glycol aginate.

Typical ointment bases may be selected from the group comprising beeswax, paraffin, cetanol, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

Typical hydrophobic ointment bases may be selected from the group comprising paraffins, vegetable oils, animal fats, synthetic glycerides, waxes, lanolin, and liquid polyalkylsiloxanes. Typical hydrophilic ointment bases are, but not limited to, solid macrogols (polyethylene glycols).

Suitable powder components may be selected from the group comprising alginate, collagen, lactose, powder, which is able to form a gel when applied to a wound (absorbs liquid/wound exudate).

Suitable diluents and disintegrating agents may be selected from the group comprising lactose, saccharose, emdex, calcium phosphates, calcium carbonate, calcium sulphate, mannitol, starches and microcrystaline cellulose.

Suitable binding agents may be selected from the group comprising saccharose, sorbitol, gum acacia, sodium alginate, gelatine, starches, cellulose, sodium carboxymethylcellulose, methylcellulose, hydroxypropylcellulose, polyvinylpyrrolidone and polyethyleneglycol.

Typical wetting agents may be selected from the group comprising sodium laurylsulphate and polysorbate 80.

Suitable lubricants may be selected from the group comprising talc, magnesium stearate, calcium stearate, silicium oxide, precirol and polyethylenglycol.

Suitable coating agents may be selected from the group comprising hydroxypropylcellulose, hydroxypropylmethylcellulose, polyvinylpropylidone, ethylcellulose and polymethylacrylates.

Typical suppository bases may be selected from the group comprising oleum cacao, adeps solidus and polyethylenglycols.

In another aspect of the invention, the composition is for use as a dietary supplement.

A dietary supplement is defined according to the U.S. Food and Drug Administration in the Dietary Supplement Health and Education Act of 1994 (DSHEA). The DSHEA gives defines a dietary supplement as “. . . a product (other than tobacco) that is intended to supplement the diet that bears or contains one or more of the following dietary ingredients: a vitamin, a mineral, an herb or other botanical, an amino acid, a dietary substance for use by man to supplement the diet by increasing the total daily intake, or a concentrate, metabolite, constituent, extract, or combinations of these things”. . . . and “is intended for ingestion in pill, capsule, tablet, or liquid form.”

Similar definitions exist in other parts of the world, e.g. in Europe. In the present context, the definition is as defined above. Different denominations concerning “dietary supplements” are used around the world, such as “food supplements”, “neutraceuticals”, “functional foods” or simply “foods”. In the present context the term “dietary supplement” covers any such denomination or definition.

The composition of the invention comprises one or more cysteine derivative(s) of Formula I or salt(s) thereof and one or more optionally substituted aminosugar(s) or salts thereof. The aminosugar may be as defined supra, but in interesting embodiments of the invention, the aminosugar may be selected from the group consisting of glucosamine, galactosamine, derivatives and salts thereof, e.g. wherein the aminosugar is N-acetylglucosamine or N-acetylgalactosamine. A preferred composition comprises glucosamine sulfate.

Another aspect of the invention relates to the pharmacological effects observed for the chemical complexes and the compositions disclosed by the present invention. It has surprisingly been found that the chemical complex or composition of the invention exhibits an anti-inflammatory effect in the same order as seen for the non-steroidal anti-inflammatory drug, Ibuprofen. Moreover, it was demonstrated that the anti-inflammatory effect of the chemical complex or composition of the invention was dose-dependent, thus indicating that the chemical complex or composition has a direct effect on inflammation.

The anti-inflammatory activity was demonstrated in the carrageenin-induced paw oedema test in rats, which is a commonly employed method for screening and evaluation of antiinflammatory drugs (see example 282)

Thus, in a broadly sense the chemical complexes or compositions of the invention provides an anti-hypersensitivity and anti-inflammatory. The present inventor has recognised that a number of diseases or conditions relates to the inflammation provoked in the carrageenin-induced paw oedema test in the rats. Such diseases or conditions may be treated with the present complexes and compositions of the invention. In a more specific sense, the chemical complexes or compositions of the invention provide suppression of hypersensitivity reactions, suppression of inflammatory reactions, suppression of cartilage degeneration, suppression of IgE mediated allergic reactions, suppression of autoimmune reactions, reduction of pain, and suppression of cancer.

Given the pharmacological actions of a chemical complex consisting of one or more cysteine derivative(s) of Formula I or salt(s) thereof and one or more optionally substituted aminosugar(s) or salt(s) thereof, the use of a combination of one or more cysteine derivative(s) of Formula I or salt(s) thereof and one or more optionally substituted aminosugar(s) or salt(s) thereof, of a complex consisting of said combination or a composition comprising said combination for the preparation of a product for the suppression of hypersensitivity and/or suppression of inflammatory reactions in a mammal is a further aspect of the invention.

Moreover, a still further aspect relates to a method for suppression of hypersensitivity and suppression of inflammatory reactions in a mammal, comprising the administration to said mammal of an effective amount of a combination of one or more cysteine derivative(s) of Formula I or salt(s) thereof and one or more optionally substituted aminosugar(s) or salt(s) thereof, or a chemical complex comprising said combination.

As defined herein, the term “mammal” is intended to include all mammals including a human.

As used herein, the term “effective amount” relates to the effective dose to be determined by a qualified practitioner, who may titrate dosages to achieve the desired response. Factors for consideration of dose will include potency, bioavailability, desired pharmacokinetic/pharmacodynamic profiles, condition of treatment, patient-related factors (e.g. weight, health, age, etc.), presence of co-administered medications (e.g., anticoagulants), time of administration, or other factors known to a medical practitioner.

As used herein, the “term treatment” relates to treatment of symptoms or prevention of the relapse of symptoms in a person diagnosed with a disease related to inflammation, hypersensitivity, infection, cancer and/or pain.

As stated, the chemical complexes or compositions of the invention may provide suppression of hypersensitivity reactions, suppression of inflammatory reactions, suppression of cartilage degeneration, suppression of IgE mediated allergic reactions, suppression of autoimmune reactions, reduction of pain, and suppression of cancer.

In one embodiment, the suppression of inflammatory reactions is in the managing of skin diseases, e.g treatment of skin diseases such as contact dermatitis, seborrhoeic dermatitis, pruritus, nodular prurigo (prurigo nodularis hyde), urticaria, acne, rosacea, alopecia, vitiligo or psoriasis.

In another embodiment, the suppression of hypersensitivity and/or suppression of inflammatory reactions is/are for the treatment of IgE mediated allergic reactions, such as asthma, eczema (e.g. atopic dermatitis), urticaria, allergic rhinitis and/or anaphylaxis.

As stated, the complexes and compositions according to the invention are of use in the treatment of autoimmune diseases. For illustrative purposes, the treatment of autoimmune disorders relates to the treatment of Autoimmune hepatitis, Primary biliary cirrhosis, Primary sclerosing cholangitis, Autoimmune hemolytic anemias, Grave's disease, Myasthenia gravis, Type 1 Diabetes Mellitus, Inflammatory myopathies, Multiple sclerosis, Hashimoto's thyreoiditis, Autoimmune adrenalitis, Crohn's Disease, Ulcerative Colitis, Glomerulonephritis, Progressive Systemic Sclerosis (Scleroderma), Sjogren's Disease, Lupus Erythematosus, Primary vasculitis, Rheumatoid Arthritis, Juvenile Arthritis, Mixed Connective Tissue Disease, Psoriasis, Pemfigus, Pemfigoid, and Dermatitis Herpetiformis.

Thus, in one embodiment the treatment of hypersensitivity, inflammation or cartilage degeneration relates to the treatment of rheumatic disorders, e.g. rheumatoid arthritis, osteoarthritis, ankylosing spondylitis, Reiter's syndrome, psoriastic arthritis, juvenile chronic arthritis, enteropathic synovitis, infective arthritis, soft tissue rheumatism and fibromyalgia. In another embodiment, the hypersensitivity and inflammation relates to the treatment of gout. In an interesting embodiment thereof, the compositions and complexes is for the treatment of muscle pain, e.g. muscle pains in relation to arthritis.

The therapeutic action of the complexes and compositions of the invention may be relevant to diseases associated with hypersensitivity reactions or inflammation in general. Accordingly, the chemical complexes or compositions of the invention are suitable for the treatment or prevention of diseases caused by inflammation of various tissues, e.g. inflammation of the prostate, in particular prostatitis. Particularly, the treatment of hypersensitivity relates to the treatment of contact dermatitis, insect bites, allergic vasculitis, post-operative reactions, transplantation rejection (graft-versus-host disease), and so forth.

Furthermore, the complexes and the compositions of the invention may be used for the treatment of cancer. The present inventor puts forward the hypothesis that the anticancer effect is due to a combination of immunomodulating and tumour-suppressing effects of the complexes and compositions of the invention.

The use of a product combining the cysteine derivative(s) of Formula I and the optionally substituted aminosugar may be done in an array of manners of administration. The cysteine derivative(s) of Formula I and the optionally substituted aminosugar may together be comprised in a single formulation or are each individually comprised in separate formulations. Furthermore, the manner of administration may be such that the combination is administered in a simultaneous or non-simultaneous manner. Thus, a formulation containing a cysteine derivative(s) of Formula I may be administered first and another separate formulation containing an optionally substituted aminosugar may be administered simultaneously or subsequently, or in an opposite order of administration.

However, in a preferred embodiment, the cysteine derivative(s) of Formula I and the optionally substituted aminosugar are together comprised in a single formulation.

In a further preferred embodiment, the combination of a cysteine derivative(s) of Formula I and an optionally substituted aminosugar is a chemical complex as defined supra.

According to the use of a product combining a cysteine derivative(s) of Formula I and an optionally substituted aminosugar, the product may further comprise one or more therapeutically active agents.

Moreover, the product of the invention may be administered by means of oral, topical, transdermal, or parenteral administration, or combinations thereof. However, preferable manners of administration are oral and/or topical administration.

### EXAMPLES

The following Examples describe the preparation of chemical complexes of the present invention.

### General Method Examples 1-271

The cysteine derivative and the aminosugar are dissolved in as little solvent as possible and the solvent is removed by spray drying or freeze-drying. After the solvent is removed the product is a white to yellowish powder. The solvent may be any organic solvent or water or mixtures thereof.

The powder is suitable for any type of product e.g. pharmaceutical products, dietary supplements and cosmetic formulations. Non-limiting examples of such products are tablets, capsules, ointments and lotions as described above.

### Examples 1 to 17

### Molar Ratio Cysteine Derivative/Aminosugar Derivative 1:10000 (mol/mol).

| | Cysteine derivative (1 mol) | Aminosugar (10000 mol) |
|---|---|---|
| Example 1. | N-acetylcysteine | Glucosamine |
| Example 2. | N-acetylcysteine | Glucosamine HCl |
| Example 3. | Cysteine | Glucosamine sodium sulfate salt |
| Example 4. | cysteine HCl | Glucosamine 2 sulfate, free acid |
| Example 5. | N-acetyl-cysteine | β-glucuronic-acid-[1→3]-N-acetyl-β- |
| | | galactosamine-6Sulfate, Na⁺ salt |
| Example 6. | N-acetyl-cysteine | Glucosamine 2 sulfate, Na⁺ salt |
| Example 7. | Cystine | Glucosamine 3 sulfate, free acid |
| Example 8. | Homocysteine | Glucosamine 3 sulfate, K⁺ salt |
| Example 9. | Gluthatione | N-acetylglucosamine 3,6 sulfate, di Na⁺ salt |
| Example 10. | Cysteine methylester HCl | N-acetylglucosamine 3,4,6 sulfate, Na⁺ salt |
| Example 11. | S-ethyl-homocysteine | N-acetylglucosamine 3,4,6 sulfate, tri Na⁺ salt |
| Example 12. | N,S-diacetyl-cysteine | Galactosamine 3,6 sulfate, di K⁺ salt |
| | methylester | |
| Example 13. | N-isobutyryl-cysteine | Mannosamine HCl |
| Example 14. | N-acetylcysteine | N-acetylgalactosamine |
| Example 15. | N-acetylcysteine | N-acetylgalactosamine 3 sulfate, Na⁺ salt |
| Example 16. | N-acetylcysteine | β-galactose-[1→3]-β-N-acetylglucosamine- |
| | | 6sulfate |
| Example 17. | N-acetylcysteine | N-acetylgalactosamine 3 sulfate, K⁺ salt |

### Examples 18 to 33

### Molar Ratio Cysteine Derivative/Aminosugar Derivative 1:1000 (mol/mol).

| | Cysteine derivative (1 mol) | Aminosugar (1000 mol) |
|---|---|---|
| Example 18. | Cystine | Glucosamine |
| Example 19. | Homocysteine | Glucosamine HCl |
| Example 20. | Homocysteine | Dermatan sulfate Na⁺ salt (average Mw |
| | | 4.000 g/mol) |
| Example 21. | Cysteine S-sulfate | Glucosamine potassium sulfate salt |
| Example 22. | N-acetylcysteine | Mannosamine |
| Example 23. | N-acetylcysteine | Galactosamine HCl |
| Example 24. | N-acetylcysteine | β-glucuronic acid-[1→3]-β-N- |
| | | acetylglucosamine |
| Example 25. | N-acetyl-S-methylcysteine | Galactosamine sodium sulfate salt |
| Example 26. | N-acetyl-S-methylcysteine | β-glucosamin-[1→4]-β-glucosamine |
| Example 27. | S-carboxymethyl-cysteine | N-acethylgalactosamine 6 sulfate, Na⁺ salt |
| Example 28. | Cysteine | Chondroitin sulfate A, K⁺ salt (average Mw |
| | | 1000 g/mol) |
| Example 29. | S-ethyl-cysteine | N-acetylgalactosamine 6 sulfate, K⁺ salt |
| Example 30. | N-acetylcysteine | N-acetylgalactosamine 3,6 sulfate, free acid |
| Example 31. | N-acetylcysteine | N-acetylgalactosamine 3,6 sulfate, Na⁺ salt |
| Example 32. | Cystine | Hyaluronic acid (average Mw 4.000 g/mol) |
| Example 33. | N-acetylcysteine | hexa([1→4]-β-N-acetyl-D-glucosamine) |

### Examples 34 to 59

### Molar Ratio Cysteine Derivative/Aminosugar Derivative 1:100 (mol/mol).

| | Cysteine derivative (1 mol) | Aminosugar (100 mol) |
|---|---|---|
| Example 34. | N-acetylcysteine | Glucosamine |
| Example 35. | Cysteine methylester HCl | Glucosamine HCl |
| Example 36. | S-ethyl-homocysteine | Glucosamine potassium sulfate salt |
| Example 37. | N-acetylcysteine | Glucosamine 2 sulfate, free acid |
| Example 38. | N-acetylcysteine | β-N-acetyl-d-glucosamin-[1→4]-β-N-acetyl- |
| | | glucosamin, Na⁺ salt |
| Example 39. | N-isobutyryl-cysteine | Glucosamine 3 sulfate, K⁺ salt |
| Example 40. | N-acetylcysteine | Chondroitin sufate A, Na⁺ salt (average Mw |
| | | 17.000 g/mol) |
| Example 41. | S-methyl-cysteine | Glucosamine 6 sulfate, Na⁺ salt |
| Example 42. | Cysteine methylester HCl | Dermatan sulfate, K⁺ salt (average Mw |
| | | 1.000 g/mol) |
| Example 43. | Cysteine S-sulfate | Glucosamine 2,3 sulfate, free acid |
| Example 44. | N-acetylcysteine | Glucosamine 2,3 sulfate, di Na⁺ salt |
| Example 45. | S-carboxymethyl-cysteine | N-acetylglucosamine HCl |
| Example 46. | S-ethyl-cysteine | N-acetylglucosamine 3 sulfate, Na⁺ salt |
| Example 47. | N-acetylcysteine | Galactosamine 3,6 sulfate, K⁺ salt |
| Example 48. | S-ethyl-cysteine | octa(β-glucuronic acid--[1→3]-N-acetyl-β- |
| | | galactosamine-6-sulfate-[1→4]) |
| Example 49. | N-acetyl-S-methylcysteine | Galactosamine 3,6 sulfate, di K⁺ salt |
| Example 50. | N-acetylcysteine | Galactosamine 3,4,6 sulfate, di Na⁺ salt |
| Example 51. | N-acetylcysteine | Galactosamine 3,4,6 sulfate, tri Na⁺ salt |
| Example 52. | N,S-diacetyl-cysteine | N-acetylgalactosamine |
| | methylester | |
| Example 53. | N-acetylcysteine | N-acetylgalactosamine potassium sulfate salt |
| Example 54. | N-acetylcysteine | N-acetylgalactosamine HCl |
| Example 55. | Cysteine | N-acetylgalactosamine 3 sulfate, free acid |
| Example 56. | N-acetylcysteine | di(β-iduronic acid-[1→3]-N-acetyl-β- |
| | | galactosamien-4sulfate-[1→4]) |
| Example 57. | Cysteine HCl | Chitin, Na⁺ salt (average Mw 4.000 g/mol) |
| Example 58. | N-acetyl-cysteine | N-acetylgalactosamine 3 sulfate, K⁺ salt |
| Example 59. | Cysteine | β-glucuronic acid-[1→3]-N-acetyl-β- |
| | | galactosamine-4-sulfate |

### Examples 60 to 79

### Molar Ratio Cysteine Derivative/Aminosugar Derivative 1:50 (mol/mol).

| | Cysteine derivative (1 mol) | Aminosugar (50 mol) |
|---|---|---|
| Example 60. | N-acetylcysteine | Glucosamine |
| Example 61. | N-acetylcysteine | Glucosamine HCl |
| Example 62. | Cysteine | Glucosamine potassium sulfate salt |
| Example 63. | N-acetylcysteine | Keratan sulfate, Na⁺ salt (average Mw |
| | | 25.000 g/mol) |
| Example 64. | Cysteine HCl | Glucosamine 2 sulfate, free acid |
| Example 65. | Cysteine HCl | β-glucuronic acid-[1→3]-N-acetyl- |
| | | galactosamine-4sulfate |
| Example 66. | N-acetyl-cysteine | Glucosamine 2-sulfate, Na⁺ salt |
| Example 67. | N-acetylcysteine | N-acetylgalactosamine 3-sulfate, free acid |
| Example 68. | N-acetylcysteine | N-acetylgalactosamine 3 sulfate, Na⁺ salt |
| Example 69. | Cystine | N-acetylgalactosamine 4 sulfate, K⁺ salt |
| Example 70. | Homocysteine | hexa(β-glucuronic acid-[1→3]-β-N- |
| | | acetylglucosamine), Na⁺ salt |
| Example 71. | gluthatione | N-acetylgalactosamine 3,6 sulfate, Na⁺ salt |
| Example 72. | Cysteine | Chitosan, Na⁺ salt (average Mw 8.000 g/mol) |
| Example 73. | N-acetylcysteine | N-acetylgalactosamine 3,6 sulfate, di Na⁺ salt |
| Example 74. | N-acetylcysteine | N-acetylgalactosamine 3,6 sulfate, K⁺ salt |
| Example 75. | Cysteine methylester HCl | N-acetylgalactosamine 3,4,6 sulfate, K⁺ salt |
| Example 76. | gluthatione | Chondroitin sulfate A, Na⁺ salt (average Mw |
| | | 2.500 g/mol) |
| Example 77. | S-ethyl-homocysteine | N-acetylgalactosamine 3,4,6 sulfate, Na⁺ salt |
| Example 78. | N-acetylcysteine | N-acetylgalactosamine 3,4,6 sulfate, di Na⁺ |
| | | salt |
| Example 79. | N-acetylcysteine | N-acetylgalactosamine 3,4,6 sulfate, tri Na⁺ |
| | | salt |

### Examples 80 to 102

### Molar Ratio Cysteine Derivative/Aminosugar Derivative 1:2 (mol/mol).

| | Cysteine derivative (1 mol) | Aminosugar (2 mol) |
|---|---|---|
| Example 80. | N-acetylcysteine | Glucosamine 2,3 sulfate, free acid |
| Example 81. | N-acetylcysteine | Glucosamine 2,3 sulfate, di Na⁺ salt |
| Example 82. | N-acetylcysteine | Chitosan, K⁺ salt (average Mw 2000 g/mol) |
| Example 83. | N-acetylcysteine | Glucosamine 2,6 sulfate, Na⁺ salt |
| Example 84. | S-methyl-cysteine | Glucosamine 3,6 sulfate, di Na⁺ salt |
| Example 85. | Cysteine S-sulfate | Glucosamine 3,4,6 sulfate, free acid |
| Example 86. | S-methyl-cysteine | Chondroitin sulfate C, Na⁺ salt (average Mw |
| | | 11.000 g/mol) |
| Example 87. | N-acetyl-S-methylcysteine | N-acetylglucosamine |
| Example 88. | N-acetylcysteine | N-acetylglucosamine |
| Example 89. | N-acetylcysteine | N-acetylglucosamine 3 sulfate, free acid |
| Example 90. | N-acetylcysteine | tri(β-glucuronic acid-[1→3]-N-acetyl-β- |
| | | galactosamine-4-sulfate-[1→4]) |
| Example 91. | N-acetylcysteine | N-acetylglucosamine 3 sulfate, Na⁺ salt |
| Example 92. | S-carboxymethyl-cysteine | N-acetylglucosamine 6 sulfate, free acid |
| Example 93. | S-ethyl-cysteine | Galactosamine |
| Example 94. | N-acetylcysteine | Galactosamine HCl |
| Example 95. | N-acetylcysteine | Galactosamine potassium sulfate salt |
| Example 96. | N-acetylcysteine | β-iduronic acid-[1→3]-N-acetyl-β- |
| | | galactosamine-4-sulfate |
| Example 97. | N-acetylcysteine | Galactosamine 2 sulfate, free acid |
| Example 98. | S-ethyl-homocysteine | Galactosamine 2 sulfate, Na⁺ salt |
| Example 99. | N-acetylcysteine | Galactosamine 2 sulfate, K⁺ salt |
| Example 100. | S-ethyl-homocysteine | Dermatan sulfate, Na⁺ salt (average Mw |
| | | 1000 g/mol) |
| Example 101. | N-acetylcysteine | Galactosamine 3 sulfate, free acid |
| Example 102. | N-acetylcysteine | Galactosamine 3 sulfate, Na⁺ salt |

### Examples 103 to 145

### Molar Ratio Cysteine Derivative/Aminosugar Derivative 4:3 (mol/mol).

| | Cysteine derivative (4 mol) | Aminosugar (3 mol) |
|---|---|---|
| Example 103. | N,S-diacetyl-cysteine | Glucosamine |
| | methylester | |
| Example 104. | N-isobutyryl-cysteine | Glucosamine HCl |
| Example 105. | N-acetylcysteine | Glucosamine potassium sulfate salt |
| Example 106. | N-acetylcysteine | Glucosamine 2 sulfate, free acid |
| Example 107. | Cysteine | Glucosamine 3 sulfate, Na⁺ salt |
| Example 108. | N-acetylcysteine | Glucosamine 6 sulfate, K⁺ salt |
| Example 109. | N-acetyl-cysteine | Glucosamine 2,3 sulfate, di Na⁺ salt |
| Example 110. | N-acetylcysteine | octa(β-glucuronic acid--[1→3]-N-acetyl-β- |
| | | galactosamine-6-sulfate-[1→4]) |
| Example 111. | Cystine | Glucosamine 2,6 sulfate, Na⁺ salt |
| Example 112. | gluthatione | Glucosamine 3,4,6 sulfate, free acid |
| Example 113. | Cysteine methylester HCl | N-acetylglucosamine |
| Example 114. | N-acetylcysteine | N-acetylglucosamine HCl |
| Example 115. | N-acetylcysteine | N-acetylglucosamine 3 sulfate, Na⁺ salt |
| Example 116. | Cysteine methylester HCl | Keratan sulfate, Na⁺ salt (average Mw |
| | | 33.000 g/mol) |
| Example 117. | N-acetylcysteine | N-acetylglucosamine 6 sulfate, free acid |
| Example 118. | S-ethyl-homocysteine | N-acetylglucosamine 6 sulfate, K⁺ salt |
| Example 119. | N,S-diacetyl-cysteine | N-acetylglucosamine 3,6 sulfate, di Na⁺ salt |
| | methylester | |
| Example 120. | N-isobutyryl-cysteine | N-acetylglucosamine 3,4,6 sulfate, Na⁺ salt |
| Example 121. | S-methyl-cysteine | N-acetylglucosamine 3,4,6 sulfate, tri Na⁺ salt |
| Example 122. | N-acetylcysteine | Galactosamine |
| Example 123. | N-acetylcysteine | Galactosamine HCl |
| Example 124. | Cysteine S-sulfate | Galactosamine sodium sulfate salt |
| Example 125. | N-acetylcysteine | di(β-glucuronic acid-[1→3]-N-acetyl- |
| | | galactosamine-4-sulfate-[1→4]) |
| Example 126. | N-acetyl-S-methylcysteine | Galactosamine 2 sulfate, Na⁺ salt |
| Example 127. | S-ethyl-cysteine | Mannosamine 3 sulfate, K⁺ salt |
| Example 128. | N-acetylcysteine | N-acetylmannosamine |
| Example 129. | N-acetylcysteine | Galactosamine 6 sulfate, K⁺ salt |
| Example 130. | N-acetylcysteine | β-glucuronic acid-[1→3]-N-acetyl- |
| | | galactosamine-6-sulfate-[1→4]) |
| Example 131. | N-acetylcysteine | Galactosamine 2,3 sulfate, K⁺ salt |
| Example 132. | N-acetylcysteine | Galactosamine 2,6 sulfate, di Na⁺ salt |
| Example 133. | N-acetylcysteine | di(β-glucuronic acid-[1→3]-β-N-acetyl- |
| | | glucosamine-[1→4]) |
| Example 134. | N-acetylcysteine | Galactosamine 3,4,6 sulfate, tri Na⁺ salt |
| Example 135. | Cystine | N-acetylgalactosamine |
| Example 136. | Homocysteine | N-acetylgalactosamine potassium sulfate salt |
| Example 137. | gluthatione | N-acetylgalactosamine HCl |
| Example 138. | Cysteine methylester HCl | N-acetylgalactosamine 4 sulfate, K⁺ salt |
| Example 139. | S-ethyl-homocysteine | N-acetylgalactosamine 6 sulfate, free acid |
| Example 140. | Gluthatione | β-galactose-[1→3]-β-N-acetyl-glucosamine-6- |
| | | sulfate |
| Example 141. | N,S-diacetyl-cysteine | Chitin, Na⁺ salt (average Mw 17.000 g/mol) |
| | methylester | |
| Example 142. | N-acetylcysteine | N-acetylgalactosamine 3,6 sulfate, di Na⁺ salt |
| Example 143. | N-acetylcysteine | N-acetylgalactosamine 3,4,6 sulfate, K⁺ salt |
| Example 144. | N-acetylcysteine | N-acetylgalactosamine 3,4,6 sulfate, tri Na⁺ |
| | | salt |
| Example 145. | N-acetylcysteine | di([1→4]β-N-acetyl-D-glucosamine) |

### Examples 146 to 172

### Molar Ratio Cysteine Derivative/Aminosugar Derivative 50:1 (mol/mol).

| | Cysteine derivative (50 mol) | Aminosugar (1 mol) |
|---|---|---|
| Example 146. | gluthatione | Glucosamine |
| Example 147. | Cysteine methylester HCl | Glucosamine HCl |
| Example 148. | N-acetylcysteine | Glucosamine potassium sulfate salt |
| Example 149. | N,S-diacetyl-cysteine | Glucosamine 2 sulfate, free acid |
| | methylester | |
| Example 150. | N-isobutyryl-cysteine | Glucosamine 2 sulfate, Na⁺ salt |
| Example 151. | S-methyl-cysteine | N-acethylglucosamine 3,6 sulfate, di Na⁺ salt |
| Example 152. | N-acetylcysteine | N-acetylglucosamine 3,4,6 sulfate, di Na⁺ salt |
| Example 153. | N-acetylcysteine | Galactosamine 2 sulfate, Na⁺ salt |
| Example 154. | S-ethyl-homocysteine | Galactosamine 2 sulfate, K⁺ salt |
| Example 155. | N-acetylcysteine | Chondroitin sulfate, Na⁺ salt (average Mw |
| | | 12.000 g/mol) |
| Example 156. | S-methyl-cysteine | Hyaluronic. acid (average Mw 5.000 g/mol) |
| Example 157. | N-acetylcysteine | Galactosamine 3 sulfate, free acid |
| Example 158. | N-acethylcysteine | N-acetylgalactosamine 3 sulfate, K⁺ salt |
| Example 159. | N-isobutyryl-cysteine | Di(β-glucuronic acid-[1→3]-β-N- |
| | | acetylglucosamine-[1→4]) |
| Example 160. | N-acetylcysteine | N-acetylgalactosamine 4 sulfate, Na⁺ salt |
| Example 161. | Cysteine S-sulfate | N-acetylgalactosamine 4 sulfate, K⁺ salt |
| Example 162. | N-acetyl-S-methylcysteine | N-acetylgalactosamine 6 sulfate, free acid |
| Example 163. | S-carboxymethyl-cysteine | Chondroitin sulfate (average Mw 17.000 g/mol) |
| Example 164. | S-ethyl-cysteine | N-acetylgalactosamine 6 sulfate, K⁺ salt |
| Example 165. | N-acetylcysteine | N-acetylgalactosamine 3,6 sulfate, free acid |
| Example 166. | Cysteine HCl | N-acetylgalactosamine 3,6 sulfate, Na⁺ salt |
| Example 167. | N-acetyl-cysteine | N-acetylgalactosamine 3,6 sulfate, di Na⁺ salt |
| Example 168. | Cysteine HCl | Dermatan sulfate (average Mw 30.000 g/mol) |
| Example 169. | N-acetylcysteine | Di([1→4]-β-D-glucosamine) |
| Example 170. | Cystine | N-acetylgalactosamine 3,4,6 sulfate, di Na⁺ |
| | | salt |
| Example 171. | N-acethylcysteine | N-acetylgalactosamine 3,4,6 sulfate, tri Na⁺ |
| | | salt |
| Example 172. | Cystine | β-galactose-[1→3]-β-N-acetylglucosamine-6- |
| | | sulfate Na⁺ salt |

### Examples 173 to 198

### Molar Ratio Cysteine Derivative/Aminosugar Derivative 500:1 (mol/mol).

| | Cysteine derivative | |
|---|---|---|
| | (500 mol) | Aminosugar (1 mol) |
| Example 173. | N-acethylcysteine | Glucosamine |
| Example 174. | N-acethylcysteine | Glucosamine HCl |
| Example 175. | N-acetylcysteine | Chitin (average Mw 30.000 g/mol) |
| Example 176. | N-acetylcysteine | tri(β-iduronic acid-[1→3]-N-acetyl-β- |
| | | galactosamine-4-sulfate-[1→4]) Na⁺ salt |
| Example 177. | gluthatione | Glucosamine potassium sulfate salt |
| Example 178. | Cysteine methylester HCl | Glucosamine 2 sulfate, free acid |
| Example 179. | S-ethyl-homocysteine | Glucosamine 2 sulfate, Na⁺ salt |
| Example 180. | Cysteine methylester HCl | Hyaluronic acid |
| Example 181. | N,S-diacetyl-cysteine | Glucosamine 2 sulfate, K⁺ salt |
| | methylester | |
| Example 182. | N-acetylcysteine | Keratan sulfate (average Mw 30.000 g/mol) |
| Example 183. | N-acetylcysteine | Glucosamine 3 sulfate, Na⁺ salt |
| Example 184. | Cysteine methylester HCl | β-galactose-[1→3]-β-N-acetylglucosamine-6- |
| | | sulfate |
| Example 185. | N-acetylcysteine | Glucosamine 3 sulfate, K⁺ salt |
| Example 186. | N-acetylcysteine | Glucosamine 6 sulfate, free acid |
| Example 187. | N-isobutyryl-cysteine | Chondroitin sulfate A (average Mw |
| | | 33.000 g/mol) |
| Example 188. | S-methyl-cysteine | N-acetylglucosamine 3,6 sulfate, di Na⁺ salt |
| Example 189. | N-acetylcysteine | N-acetylglucosamine 3,4,6 sulfate, di Na⁺ salt |
| Example 190. | N-acetylcysteine | N-acetylglucosamine 3,4,6 sulfate, Na⁺ salt |
| Example 191. | N-acetylcysteine | N-acetylglucosamine 3,4,6 sulfate, di Na⁺ salt |
| Example 192. | Cysteine S-sulfate | Chondroitin sulfate C (average Mw |
| | | 15.000 g/mol) |
| Example 193. | N-acetyl-S-methylcysteine | Galactosamine 3,6 sulfate, K⁺ salt |
| Example 194. | N-acethylcysteine | Galactosamine 3,6 sulfate, di K⁺ salt |
| Example 195. | S-carboxymethyl-cysteine | tetra(β-glucuronic acid-[1→3]-N-acetyl-β- |
| | | galactosamine-4-sulfate-[1→4]) |
| Example 196. | S-ethyl-cysteine | Galactosamine 3,4,6 sulfate, tri Na⁺ salt |
| Example 197. | N-acetylcysteine | N-acetylgalactosamine |
| Example 198. | S-ethyl-cysteine | Chitosan (average Mw 50.000 g/mol) |

### Examples 199 to 215

### Molar Ratio Cysteine Derivative/Aminosugar Derivative 5000:1 (mol/mol).

| | Cysteine derivative (5000 mol) | Aminosugar (1 mol) |
|---|---|---|
| Example 199. | N-acetylcysteine | Glucosamine |
| Example 200. | Cysteine | Glucosamine HCl |
| Example 201. | cysteine HCl | Glucosamine potassium sulfate salt |
| Example 202. | Cysteine | Chondroitin sulfate B (average Mw |
| | | 60.000 g/mol) |
| Example 203. | N-acetylcysteine | Hyaluronic acid |
| Example 204. | N-acetyl-cysteine | Galactosamine |
| Example 205. | gluthatione | Galactosamine HCl |
| Example 206. | gluthatione | Chondroitin sulfate A (average Mw |
| | | 10.000 g/mol) |
| Example 207. | Cysteine methylester HCl | Galactosamine sodium sulfate salt |
| Example 208. | Cysteine methylester HCl | β-glucuronic acid-[1→3]-N-acetyl- |
| | | galactosamine-4-sulfate |
| Example 209. | S-ethyl-homocysteine | N-acetylgalactosamine 6 sulfate, Na⁺ salt |
| Example 210. | N-acetylcysteine | N-acetylgalactosamine 6 sulfate, K⁺ salt |
| Example 211. | N-acetylcysteine | hexa([1→4)-β-d-glucosamine) |
| Example 212. | N-acetylcysteine | Carboxylmethyl Chitosan (average Mw |
| | | 12.000 g/mol) |
| Example 213. | N-acetylcysteine | N-acetylgalactosamine 3,6 sulfate, free acid |
| Example 214. | N-acetylcysteine | N-acetylgalactosamine 3,6 sulfate, Na⁺ salt |
| Example 215. | S-ethyl-homocysteine | Chondroitin sulfate A (average Mw |
| | | 10.000 g/mol) |

### Examples 216 to 236

### Molar Ratio Cysteine Derivative/Aminosugar Derivative 10000:1 (mol/mol).

| | Cysteine derivative (10000 mol) | Aminosugar (1 mol) |
|---|---|---|
| Example 216. | N-acetylcysteine | Glucosamine 2,3 sulfate, di Na⁺ salt |
| Example 217. | N-acetylcysteine | Glucosamine 2,6 sulfate, Na⁺ salt |
| Example 218. | N,S-diacetyl-cysteine | Glucosamine 3,6 sulfate, di Na⁺ salt |
| | methylester | |
| Example 219. | N-isobutyryl-cysteine | Glucosamine 3,4,6 sulfate, free acid |
| Example 220. | N,S-diacetyl-cysteine | Chondroitin sulfate (average Mw |
| | methylester | 50.000 g/mol) |
| Example 221. | N-acetylcysteine | Keratan sulfate (average Mw 25.000 g/mol) |
| Example 222. | S-methyl-cysteine | N-acetylglucosamine |
| Example 223. | Cysteine S-sulfate | N-acetylglucosamine HCl |
| Example 224. | N-acetyl-S-methylcysteine | N-acetylglucosamine 3 sulfate, free acid |
| Example 225. | S-carboxymethyl-cysteine | N-acetylglucosamine 3 sulfate, Na⁺ salt |
| Example 226. | S-ethyl-cysteine | N-acetylglucosamine 6 sulfate, free acid |
| Example 227. | N-acetylcysteine | N-acetylglucosamine 6 sulfate, K⁺ salt |
| Example 228. | S-ethyl-cysteine | Hyaluronic acid |
| Example 229. | N-acetylcysteine | Galactosamine HCl |
| Example 230. | N,S-diacetyl-cysteine | Galactosamine potassium sulfate salt |
| | methylester | |
| Example 231. | N-isobutyryl-cysteine | N-acetylgalactosamine 6 sulfate, K⁺ salt |
| Example 232. | S-methyl-cysteine | hyaluraonic acid disaccharide |
| Example 233. | Cysteine S-sulfate | Chitosan (average Mw 50.000 g/mol) |
| Example 234. | N-acetyl-S-methylcysteine | Penta(β-glucuronic acid-[1→3]-N-acetyl-β- |
| | | galactosamine-4-sulfate-[1→4]) |
| Example 235. | S-carboxymethyl-cysteine | Dermatan sulfate (average Mw |
| | | 33.000 g/mol) |
| Example 236. | S-ethyl-cysteine | di([1→4]-β-N-acetyl-D-glucosamin) |

### Examples 237 to 253

### Weight Ratio Cysteine Derivative/Aminosugar Derivative 1:3 (g/g).

| | Cysteine derivative (1000 g) | Aminosugar (3000 g) |
|---|---|---|
| Example 237. | N-acetylcysteine | Glucosamine |
| Example 238. | N-acetylcysteine | Glucosamine HCl |
| Example 239. | N-acetylcysteine | Glucosamine potassium sulfate salt |
| Example 240. | N-acetylcysteine | Glucosamine 2 sulfate, free acid |
| Example 241. | N-acetylcysteine | Chondroitin A sulfate (average Mw |
| | | 30.000 g/mol) |
| Example 242. | N-acetylcysteine | Glucosamine 2 sulfate, Na⁺ salt |
| Example 243. | N,S-diacetyl-cysteine | Glucosamine 2 sulfate, K⁺ salt |
| | methylester | |
| Example 244. | N-isobutyryl-cysteine | Galactosamine |
| Example 245. | S-methyl-cysteine | Galactosamine HCl |
| Example 246. | Cysteine S-sulfate | Galactosamine potassium sulfate salt |
| Example 247. | N-acetyl-S-methylcysteine | Galactosamine 2 sulfate, free acid |
| Example 248. | S-carboxymethyl-cysteine | Dermatan sulfate (average Mw 15.000 g/mol) |
| Example 249. | S-ethyl-cysteine | Galactosamine 2 sulfate, K⁺ salt |
| Example 250. | N-acetylcysteine | N-acetylgalactosamine |
| Example 251. | N-acetylcysteine | N-acetylgalactosamine potassium sulfate salt |
| Example 252. | N-acetylcysteine | N-acetylgalactosamine HCl |
| Example 253. | N-acetylcysteine | di([1→4]-β-N-acetyl-D-glucosmaine) |

### Examples 254 to 271

### Weight Ratio Cysteine Derivative/Aminosugar Derivative 1: 10 (g/g).

| | Cysteine derivative (100 g) | Aminosugar (1000 g) |
|---|---|---|
| Example 254. | N-acetylcysteine | N-acetylgalactosamine 4 sulfate, K⁺ salt |
| Example 255. | N-acetylcysteine | N-acetylgalactosamine 6 sulfate, Na⁺ salt |
| Example 256. | N-acetyl-cysteine | N-acetylgalactosamine 6 sulfate, K⁺ salt |
| Example 257. | Cystine | N-acetylgalactosamine 3,6 sulfate, Na⁺ salt |
| Example 258. | Homocysteine | N-acetylgalactosamine 3,6 sulfate, di Na⁺ salt |
| Example 259. | gluthatione | Glucosamine 2,6 sulfate, Na⁺ salt |
| Example 260. | Cysteine methylester HCl | Glucosamine 3,6 sulfate, di Na⁺ salt |
| Example 261. | S-ethyl-homocysteine | Glucosamine 3,4,6 sulfate, free acid |
| Example 262. | N,S-diacetyl-cysteine | N-acetylglucosamine |
| | methylester | |
| Example 263. | N-isobutyryl-cysteine | N-acetylglucosamine HCl |
| Example 264. | S-methyl-cysteine | N-acetylglucosamine 3 sulfate, free acid |
| Example 265. | N-acetylcysteine | Keratan sulfate (average Mw 40.000 g/mol) |
| Example 266. | N-acetylcysteine | di([1→4]-β-N-acetyl-D-glucosamin) |
| Example 267. | N-acetylcysteine | N-acetylglucosamine 3 sulfate, Na⁺ salt |
| Example 268. | Cysteine S-sulfate | N-acetylglucosamine 6 sulfate, free acid |
| Example 269. | N-acetyl-S-methylcysteine | N-acetylglucosamine 6 sulfate, K⁺ salt |
| Example 270. | S-carboxymethyl-cysteine | N-acetylglucosamine 3,6 sulfate, di Na⁺ salt |
| Example 271. | S-ethyl-cysteine | β-glucuronic acid-[1→3]-N-acetyl- |
| | | galactosamine-4-sulfate |

### General Method Examples 272-280

Pharmaceutical compositions according to the invention are prepared. A quantity of cysteine derivative and the aminosugar derivative are transferred to a hard gelatine capsule.

### Examples 272 to 280

### Capsule 500 mg, Molar Ratio Cysteine Derivative/Aminosugar Derivative 3:4

| | Cysteine derivative | |
|---|---|---|
| | quantity | Aminosugar quantity |
| Example 272. | N-acetylcysteine 132 g | Glucosamine sulfate 368 g |
| Example 273. | N-acetylcysteine 178 g | N-acetylglucosamine 322 g |
| Example 274. | N-acetylcysteine 181 g | Galactosamine HCl 319 g |
| Example 275. | N-acetylcysteine 152 g | Glucosamine 2 sulfate, Na⁺ salt 348 g |
| Example 276. | Cysteine HCl 190 g | Galactosamine HCl 310 g |
| Example 277. | Gluthatione free acid 225 g | Glucosamine 2 sulfate, Na⁺ salt 275 g |
| Example 278. | N-acetylcysteine 29 g | Chondroitin sulfate A (2000 g/mol) 471 g |
| Example 279. | N-acetylcysteine 95 g | 3-glucuronic acid-[1→3]-N-acetyl- |
| | | galactosamine-4-sulfate 405 g |
| Example 280. | Homocysteine 101 g | Hyaluronic acid disaccharide Na⁺ salt 399 g |

### Example 281

In a small preliminary clinical investigation three persons administered a pharmaceutical composition according to the invention. The composition was an aqueous solution (100 mg/ml) of compound 105, which is the complex of the invention prepared according to example 105.

One patient (female) was 68 years old and had suffered from tendonitis of the arm for eight months. The symptoms were chronic muscle pain and some degree of immobility of the arm. Treatment with normal doses of celecoxib or ibuprofen only had a limited symptomatic effect and had therefore been given up.

The composition of the invention was administered orally corresponding to 3000 mg compound 105 twice a day. After two weeks a clear improvement of the pain was observed and after three weeks all symptoms had disappeared. After a further week of treatment the treatment was discontinued. Three months after stopping the treatment the symptoms had not reappeared and the patient was considered cured.

Another patient (male) was 67 years old and had suffered from osteoarthritis of the hips and knees for ten years. For some periods the patient had been treated with celecoxib, paracetamol and/or codein to control the pain. The main symptoms were pain and immobility of the joints. The composition of the invention was administered orally corresponding to 3000 mg compound 105 twice a day. After two weeks a clear improvement of the pain was observed and gradually the mobility of joints was also improved. After 6 months of treatment the symptoms were still significantly reduced and the patient stable.

Another patient (male) was 34 years old and had a knee injury involving cartilage damage. The symptoms were pain and immobility of the knee joint. The composition of the invention was administered orally corresponding to 3000 mg compound 105 twice a day. After one week a clear improvement of the pain was observed and gradually the mobility of joints was also improved. After two weeks the symptoms had gone and the treatment was stopped. During the next two days the symptoms started reappearing. The treatment with the composition of the invention was therefore continued and after two days the symptoms had completely gone again.

### Example 282

Objective

The objective of this study is to assess the effect of complexes or compositions of the invention in the carrageenin-induced paw oedema test in the rat, a commonly employed method for screening and evaluation of antiinflammatory drugs. Carrageenin, the phlogistic agent of choice for testing antiinflammatory drugs, is a mucopolysaccharide derived from Irish sea moss, Chondrus. Ibuprofen is used as a positive control.

Materials and Methods

Test Article and Vehicle

The test article is the complex of the invention prepared according to example 105 (Compound 105 in the following). Compound 105 and Ibuprofen are obtained from Astion A/S, Denmark.

The test article is dissolved in milli-Q water. 0.9% NaCl solution is used as vehicle.

Animals

The study is performed in male SPF Sprague Dawley rats of the stock Mol:SPRD from M & B A/S, Tornbjergvej 40, DK-4623 Lille Skensved, Denmark. At start of the acclimatisation period the rats are in the weight range of 80-100 g.

An acclimatisation period of approximately 5-8 days is allowed in order to reject animals in poor condition or at extreme weights.

Housing

The study takes place in an animal room provided with filtered air. The temperature in the room is set at 21-23° C. and the relative humidity to >50%. The room is illuminated to give a cycle of 12 hours light and 12 hours darkness. Light is on from 06.00 till 18.00 h.

The animals are housed in Macrolon type III cages (40×25×14 cm) six in each cage. The cages will be cleaned and the bedding changed at least once a week. The animal room is cleaned and disinfected with Diversol Bx.

Bedding

The bedding is sawdust (Tapvei 4HV) from Tapvei Oy, 73620 Kortteinen, Finland.

Diet

A complete pelleted rodent diet “Altromin 1314” from Chr. Petersen, DK-4100 Ringsted, is available ad libitum.

Drinking Water

The animals will have free access to bottles with domestic quality drinking water added citric acid to pH 3.

Animal Randomisation and Allocation

On the day of arrival the animals will be randomly allocated to groups, each of 12 rats.

Animal and Cage Identification

Each animal is identified by punched earmarks. Each cage is marked with study number 2022, cage number, group number and animal numbers.

Body Weight

The animals are weighed on days −2 and 0 of dosing.

Dosing

All doses of Compound 105 are administered intraperitoneally in volumes of 20 ml per kg body weight, once daily on day −2 and −1 to groups 2,3 and 4 only and on day 0 to groups 2-6, 0-5 minutes before injection of carrageenin into the foot on day 0. Ibuprofen and vehicle are administered orally by gavage in volumes of 20 ml/kg body weight on day 0, 0-5 minutes before injection of the carrageenin into the foot.

The groups, dose levels and animal numbers are as follows:

| Group | Test article | Dose, mg/kg | Animal numbers |
|---|---|---|---|
| 1 | Vehicle-control | — | 1-12 |
| 2 | Compound 105 | 100¤ | 13-24 |
| 3 | Compound 105 | 333¤ | 25-36 |
| 4 | Compound 105 | 1000¤ | 37-48 |
| 5 | Compound 105 | 333 | 49-60 |
| 6 | Compound 105 | 1000 | 61-72 |
| 7 | Ibuprofen | 50 | 73-84 |
| 8 | Ibuprofen | 150 | 85-96 |

¤ = per administration—these doses will also be given on day −2 and −1.

Carrageenin (from Sigma) is prepared as a 1% suspension in sterile 0.9% NaCl-solution. A volume of 0.1 ml is injected through a 25-gauge needle into the plantar tissue of the right hind paw of the rats within 5 minutes after treatment with the test articles.

Measurements

Immediately before the dosing and carrageenin injection and three and five hours later the foot volume is measured using a plethysmometer LE 7500 from Letica Scientific Instruments, Spain.

Clinical Signs

All visible signs of ill health and any behavioural changes is recorded daily during the study. Any deviation from normal is recorded with respect to time of onset, duration and intensity.

Findings

After three hours an inhibition of 40, 63 and 50% of the paw oedema was seen after 100, 333, and 1000 mg/kg Compound 105 given for three days, respectively. After Compound 105 administered only once at doses of 333 mg/kg and 1000 mg/kg an inhibition of 43 and 37% was seen. Ibuprofen at dose levels of 50 and 150 mg/kg inhibited 37 and 57% respectively. After 5 hours inhibitions of 45, 77 and 68% was seen after 100, 333 and 1000 mg/kg Compound 105 given for three days respectively. After 5 hours Compound 105 administered only once at doses of 333 mg/kg and 1000 mg/kg inhibited 74 and 77%, respectively, while the two doses of Ibuprofen inhibited 77 and 97%, respectively.

Interpretation

The data imply that Compound 105 had a dose-dependent anti-inflammatory effect, which was maximal at 333 mg/kg and of the same size of order as the effect seen after 50 mg/kg Ibuprofen.

1. A chemical complex consisting of:
i) one or more cysteine derivative(s) of Formula I or salt(s) thereof; and
ii) one or more optionally substituted aminosugar(s) or salt(s) thereof. 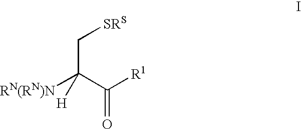
wherein R^{N} is independently selected from the group consisting of hydrogen, optionally substituted C₁-C₈-alkyl, optionally substituted C₂-C₁₀-alkenyl, optionally substituted C₂-C₁₀-alkynyl, optionally substituted C₃-C₇-cycloalkyl, and optionally substituted C₁-C₈-acyl;
R¹ is selected from the group consisting of OR3, SR3, halogen and N(RN)RN; and
R^{S} is selected from the group consisting of hydrogen, sulphate, optionally substituted C₁-C₆-alkyl, optionally substituted C₁-C₆-alkenyl, optionally substituted C₂-C₆-alkynyl, optionally substituted C₁-C₈-acyl, optionally substituted C₃-C₇-cycloalkyl, a cysteine derivative according to formula I, and two or more cysteine derivative(s) of formula I linked by S—S linkages.

2. The chemical complex according to claim 1, wherein said aminosugar is an aminosugar derivative of a monosaccharide.

3. The chemical complex according to claim 2, wherein said aminosugar derivative of a monosaccharide is selected from the group consisting of glucosamine, galactosamine, mannosamine, derivatives and salts thereof.

4. The chemical complex according to claim 1, wherein said aminosugar is an aminosugar derivative of an oligosaccharide.

5. The chemical complex according to claim 1, wherein said aminosugar is an aminosugar derivative of a polysaccharide.

6. The chemical complex according to claim 5, wherein said aminosugar derivative of a polysaccharide is selected from the group consisting of chitin, chitosan, carboxymethyl-chitosan, chondroitin sulfate, heparin, heparan sulfate, keratan sulfate and hyaluronic acid.

7. The chemical complex according to claim 3, wherein said aminosugar derivative is selected from the group consisting of glucosamine sulfate, glucosamine hydrochloride, N-acetylglucosamine, galactosamine sulfate, galactosamine hydrochloride, N-acetylgalactosamine, mannosamine sulfate, mannosamine hydrochloride or N-acetylmannosamine and salts thereof.

8. The chemical complex according to claim 3, wherein the aminosugar is glucosamine sulfate or a salt thereof.

9. The chemical complex according to claim 1, wherein the cysteine derivative(s) of Formula I or salt(s) thereof is selected from the group consisting of cysteine, Na-acetylcysteine, cystine, homocysteine, cysteine methylester, S-ethyl-cysteine, N,S-isobuturyl-cysteine, S-carboxymethyl-cysteine, S-ethyl-homocysteine, S-methyl-cysteine, Cysteine S-sulfate, N,S-diacetyl-cysteine methylester, N-acetyl-S-methylcysteine and salts thereof.

10. The chemical complex according to claim 1, wherein the cysteine derivative of Formula I or salt(s) thereof and the one or more optionally substituted aminosugar(s) or salt(s) thereof are present in a molar ratio of between about 1:10000 to 10000:1, preferably of about 1:1000 to 1000:1, more preferably of about 1:100 to 100:1, even more preferably of about 1:10 to 10:1 or of about 1:5 to 5:1, most preferably of about 1:2 to 2:1 or 1:1.

11. The chemical complex according to claim 1, wherein the cysteine derivative of Formula I or salt(s) thereof and the one or more optionally substituted aminosugar(s) or salt(s) thereof are present in a mass ratio of between about 1:10000 to 10000:1 preferably of about 1:1000 to 1000:1, more preferably of about 1:100 to 100:1, even more preferably of about 1:10 to 10:1 or of about 1:5 to 5:1, most preferably of about 1:2 to 2:1 or 1:1.

12. A composition comprising:
i) one or more cysteine derivative(s) of Formula I or salts thereof; and
ii) one or more optionally substituted aminosugar or salts thereof; and
iii) one or more acceptable excipient(s) or carrier(s), 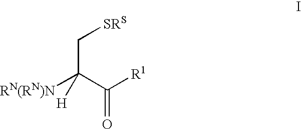
wherein R^{N} is independently selected from the group consisting of hydrogen, optionally substituted C₁-C₈-alkyl, optionally substituted C₂-C₁₀-alkenyl, optionally substituted C₂-C₁₀-alkynyl, optionally substituted C₃-C₇-cycloalkyl, and optionally substituted C₁-C₈-acyl;
R¹ is selected from the group consisting of OR3, SR3, halogen and N(RN)RN; and
R^{S} is selected from the group consisting of hydrogen, sulphate, optionally substituted C₁-C₆-alkyl, optionally substituted C₁-C₆-alkenyl, optionally substituted C₂-C₆-alkynyl, optionally substituted C₁-C₈-acyl, optionally substituted C₃-C₇-cycloalkyl, a cysteine derivative according to Formula I, and two or more cysteine derivative(s) of Formula I linked by S—S linkages, with the proviso that the composition is essentially free of Vitamin C.

13. The composition according to claim 12, wherein composition does not further comprise a non-steroidal antiinflammatory agent.

14. The composition according to claim 12, with the proviso that the composition is essentially free of a magnesium salt selected from the group consisting of magnesium ascorbate, magnesium L-acetylcysteinate, magnesium N-thioctyltaurate, magnesium taurate, magnesium citrate and magnesium oxide.

15. The composition according to claims 12, with the proviso that the composition does not contain vitamin C.

16. The composition according to claim 12, wherein the one or more acceptable excipient(s) does not include a magnesium salt selected from the group consisting of magnesium ascorbate, magnesium L-acetylcysteinate, magnesium N-thioctyltaurate, magnesium taurate, magnesium citrate and magnesium oxide.

17. The composition according to claim 12, wherein the one or more acceptable excipient(s) does not include vitamin C and a magnesium salt selected from the group consisting of magnesium ascorbate, magnesium L-acetylcysteinate, magnesium N-thioctyltaurate, magnesium taurate, magnesium citrate and magnesium oxide.

18. The composition according to claim 12, wherein said aminosugar is an aminosugar derivative of a monosaccharide.

19. The composition according to claim 18, wherein said aminosugar derivative of a monosaccharide is selected from the group consisting of glucosamine, galactosamine, mannosamine, derivatives and salts thereof.

20. The composition according to claim 12, wherein said aminosugar is an aminosugar derivative of an oligosaccharide.

21. The composition according to claim 12, wherein said aminosugar is an aminosugar derivative of a polysaccharide.

22. The composition according to claim 21, wherein said aminosugar derivative of a polysaccharide is selected from the group consisting of chitin, chitosan, carboxymethyl-chitosan, chondroitin sulfate, heparin, heparan sulfate, keratan sulfate and hyaluronic acid.

23. The composition according to claim 19, wherein said aminosugar derivative is selected from the group consisting glucosamine sulfate, glucosamine hydrochloride, N-acetylglucosamine, galactosamine sulfate, galactosamine hydrochloride, N-acetylgaiactosamine, mannosamine sulfate, mannosamine hydrochloride or N-acetylmannosamine and salts thereof.

24. The composition according to claim 19, wherein the aminosugar is a glucosamine sulfate or a salt thereof.

25. The composition according to claim 12, wherein the cysteine derivative(s) of Formula I or salt(s) thereof is selected from the group consisting of cysteine, Na-acetylcysteine, cystine, homocysteine, cysteine methylester, S-ethyl-cysteine, N,S-isobuturyl-cysteine, S-carboxymethyl-cysteine, S-ethyl-homocysteine, S-methyl-cysteine, Cysteine S-sulfate, N,S-diacetyl-cysteine methylester, N-acetyl-S-methylcysteine and salts thereof.

26. The composition according to claim 12, wherein the cysteine derivative of Formula I or salt(s) thereof and the one or more optionally substituted aminosugar(s) or salt(s) thereof are present in a molar ratio of between about 1:10000 to 10000:1, preferably of about 1:1000 to 1000:1, more preferably of about 1:100 to 100:1, even more preferably of about 1:10 to 10:1 or of about 1:5 to 5: 1, most preferably of about 1:2 to 2:1 or 1:1.

27. The composition according to claim 12, wherein the cysteine derivative of Formula I or salt(s) thereof and the one or more optionally substituted aminosugar(s) or salt(s) thereof are present in a mass ratio of between about 1:10000 to 10000:1, preferably of about 1:1000 to 1000:1, more preferably of about 1:100 to 100:1, even more preferably of about 1:10 to 10:1 or of about 1:5 to 5:1, most preferably of about 1:2 to 2:1 or 1:1

28. The composition according to claim 12 comprising
i) a complex as defined in any one of claims 1 to 11; and optionally
iii) one or more acceptable excipient(s) or carrier(s)

29. The composition according to claim 12 formulated as a pharmaceutical composition for oral, topical, transdermal, or parenteral administration.

30. The composition according to claim 29 formulated for oral or topical administration.

31. The composition according to claim 29 formulated for topical administration.

32. A method for suppression of hypersensitivity and suppression of inflammatory reactions in a mammal, comprising the administration to said mammal of an effective amount of a combination of a cysteine derivative of Formula I or salt(s) thereof and one or more optionally substituted aminosugar(s) or salt(s) thereof, or a chemical complex comprising said combination.

33. The method according to claim 32, wherein the suppression of hypersensitivity and/or suppression of inflammatory reactions is/are for the treatment of a rheumatic disease.

34. The method according to claim 33, wherein the rheumatic disease is selected from the group consisting of rheumatoid arthritis, osteoarthritis, ankylosing spondylitis, Reiter's syndrome, psoriastic arthritis, juvenile chronic arthritis, enteropathic synovitis, infective arthritis, soft tissue rheumatism and fibromyalgia.

35. The method according to claim 32, wherein the suppression of hypersensitivity and/or suppression of inflammatory reactions is/are for chondroprotection or repair of articular cartilage.

36. The method according to claim 32, wherein the suppression of hypersensitivity and/or suppression of inflammatory reactions is/are for the treatment of a skin disease.

37. The method according to claim 36, wherein the skin disease is selected from the group consisting of atopic dermatitis, contact dermatitis, seborrhoeic dermatitis, pruritus, nodular prurigo (prurigo nodularis hyde), urticaria, acne, rosacea, alopecia, vitiligo and psoriasis.

38. The method according to claim 32, wherein the suppression of hypersensitivity and/or suppression of inflammatory reactions is/are for the treatment of IgE mediated allergic reactions

39. The method according to claim 32, wherein the suppression of hypersensitivity and/or suppression of inflammatory reactions is/are for the treatment of diseases and disorders selected from the group consisting of asthma, allergic rhinitis, allergic conjunctivitis and anaphylaxis.

40. The method according to claim 32, wherein the suppression of hypersensitivity and/or suppression of inflammatory reactions is/are for the treatment of an autoimmune disease and/or a chronic inflammatory disease.

41. The method according to claim 40, wherein the suppression of hypersensitivity and/or suppression of inflammatory reactions is/are for the treatment of diabetes, Crohn's disease, lupus erythematosus, Scleroderma, Sjo\ddot{g}ren's syndrome, Graves' disease, Pernicious anemia, autoimmune hepatitis, pemphigus vulgaris, pemphigus foliaceus, bullous pemphigoid, Myasthenia gravis and rheumatoid arthritis.

42. The method according to claim 32, wherein the cysteine derivative of Formula I or salt(s) thereof and one or more optionally substituted aminosugar(s) or salt(s) thereof are together comprised in a single formulation or are each individually comprised in separate formulations.

43. The method according to claim 42, wherein the separate formulations are administered in a simultaneous or non-simultaneous manner.

44. The method according to claim 42, wherein the cysteine derivative of Formula I or salt(s) thereof and one or more optionally substituted aminosugar(s) or salt(s) thereof are together comprised in a single formulation.

45. The method according to any of claim 42, wherein the single formulation or separate formulations are administered by means of oral, topical, transdermal, or parenteral administration, or combinations thereof

46. The method according to claim 45, wherein the single formulation or separate formulations are administered by means of oral administration.

47. The use according to claim 45, wherein the single formulation or separate formulations are administered by means of topical administration.
